(19) United States
(12) Patent Application Publication
(10) Pub. No.: US 2024/0184119 A1
(43) Pub. Date: Jun. 6, 2024

(54) WEARABLE DEVICE INCLUDING AN IMAGE DISPLAY MODULE

(71) Applicant: Samsung Display Co., LTD., Yongin-si (KR)

(72) Inventors: Tae Hee LEE, Yongin-si (KR); Kyung Ho KIM, Yongin-si (KR); Young Seok SEO, Yongin-si (KR); Joo Woan CHO, Yongin-si (KR); Byeong Hwa CHOI, Yongin-si (KR)

(21) Appl. No.: 18/447,803

(22) Filed: Aug. 10, 2023

(30) Foreign Application Priority Data
Dec. 2, 2022 (KR) 10-2022-0166368

(51) Int. Cl. G02B 27/01; A61F 9/00; G02B 27/00; G02B 27/09

(52) U.S. Cl. CPC G02B 27/0172; A61F 9/0026; G02B 27/0093; G02B 27/0961; G02B 2027/0138; G02B 2027/014

(57) ABSTRACT

A wearable device includes: a main frame configured to be mounted on a user's body; an image display module on the main frame and configured to display an image; a lens frame on an image display surface of the image display module, the lens frame being configured to refract image display light emitted from the image display module; a multi-channel lens forming an emission path of the image display light refracted by the lens frame for each of a plurality of channels; and an eyeball protection module on the main frame and configured to spray air, moisture, or a tear solution to the user's left and right eyes.

### CROSS-REFERENCE TO RELATED APPLICATION

This application claims priority to and the benefit of Korean Patent Application No. 10-2022-0166368, filed on Dec. 2, 2022, in the Korean Intellectual Property Office, the entire content of which is hereby incorporated by reference.

### BACKGROUND

### 1. Field

Aspects of embodiments of the present disclosure relate to a wearable device including an image display module.

### 2. Description of the Related Art

The importance of display devices has increased with the development of multimedia. Accordingly, various types of display devices, such as a liquid crystal display (LCD) and an organic light emitting display (OLED), are in use.

From among the various types and applications of display devices, there are electronic devices provided in a form in which they may be worn on the body. Such electronic devices are commonly referred to as wearable devices. The wearable device may be directly worn on the body, and thus, portability and accessibility of a user may be improved.

A head mounted display device that may be mounted on a wearer's head is one example of a wearable device. The head mounted display device may be classified into a see-through type head mounted device that provides augmented reality (AR) and a see-closed type head mounted device that provides virtual reality (VR).

### SUMMARY

Embodiments of the present disclosure provide a wearable device including an image display module that can protect a user's eyes and reduce the user's eye fatigue.

Embodiments of the present disclosure also provide a wearable device capable of inducing a user to blink and configured to display a message according to an increase in the user's fatigue by monitoring a user's blink period and eyeball temperature.

However, aspects and features of the present disclosure are not restricted to those set forth herein. The above and other aspects and features of the present disclosure will become more apparent to one of ordinary skill in the art to which the present disclosure pertains by referencing the detailed description of the present disclosure given below.

According to an embodiment of the disclosure, a wearable device includes: a main frame configured to be mounted on a user's body; an image display module on the main frame and configured to display an image; a lens frame on an image display surface of the image display module, the lens frame being configured to refract image display light emitted from the image display module; a multi-channel lens forming an emission path of the image display light refracted by the lens frame for each of a plurality of channels; and an eyeball protection module on the main frame and configured to spray air, moisture, or a tear solution to the user's left and right eyes.

The eyeball protection module may include: first and second blink sensing units configured to sense blink motions of the user's left and right eyes in real time and to generate and outputting blink sensing signals of the user's left and right eyes; first and second temperature detection units configured to sense temperatures of the user's left and right eyes in real time and to output temperature sensing signals of the user's left and right eyes; first and second spray driving units configured to spray the air, water, or tear solution to the user's left and right eyes to induce user's blink motions; and a blink control unit configured to control spray driving of the first and second spray driving units based on at least one of the blink sensing signals of the user's left and right eyes and the temperature sensing signals for the user's left and right eyes.

The first and second blink sensing unit may include: an image sensor configured to capture images of the user's left and right eyes in real time; an image analyzer configured to compare the captured eyeball images with each other in units of at least one frame and to detect eyelid images as a comparison result; and a blink sensing signal output unit configured to generate blink sensing signals according to the detection of the eyelid images and to supply the blink sensing signals to the blink control unit.

The first and second temperature detection units may include: a contactless thermometer configured to sense the temperatures of the user's left and right eyes in real time; and an analog-to-digital converter configured to transmit the temperature sensing signals corresponding to the sensed temperatures of the user's left and right eyes to the blink control unit.

The first and second spray driving units may include: a storage capsule for storing the air, water, or tear solution; and a spray pump configured to spray the air, water, or tear solution to the user's left and right eyes during a period in which a spray control signal is input from the blink control unit.

The blink control unit may be configured to: detect a number of inputs of the blink sensing signals of the user's left and right eyes in units of a reference time;

compare the number of inputs of the blink sensing signals for each reference time unit with a reference value for each reference time; and when the number of inputs of the blink sensing signals for each reference time unit is determined to be smaller than the reference value for each reference time, transmit spray control signals to the first and second spray driving units.

When the number of inputs of the blink sensing signals for each reference time unit is determined to be smaller than the reference value for each reference time unit, the blink control unit may be configured to transmit an eyeball protection operation execution notification message to a display control unit of the image display module to allow the eyeball protection operation execution notification message to be displayed on a display panel of the image display module.

The blink control unit may be configured to: receive the temperature sensing signals of the user's left and right eyes in real time through the first and second temperature detection units; detect temperature values of the user's left and right eyes corresponding to the temperature sensing signals of the user's left and right eyes using a memory or a register; compare the temperature values of the user's left and right eyes with a threshold temperature value; and when the temperature values of the user's left and right eyes is greater than the threshold temperature value, transmit spray control signals to the first and second spray driving units.

The eyeball protection module may further include a use time detection unit configured to monitor an image display period of a display panel in the image display module and to accumulate and output information on a gaze period during which a user gazes at a displayed image, and the blink control unit may be configured to: receive the accumulated information on the gaze period during which the user gazes at the displayed image from the use time detection unit and compare the information on the gaze period of the user with use time reference information for each step; and when the information on the gaze period of the user is the same as the use time reference information for each step, transmit spray control signals to the first and second spray driving unit.

The lens frame may include first and second lens frames respectively corresponding to positions of the user's left and right eyes, and the first and second lens frames may be configured to refract the image display light emitted from the image display surface of the image display module in a front direction at an angle in an outer direction or an outer circumferential direction as compared with the front direction and to emit the refracted image display light to rear surfaces of first and second multi-channel lenses, respectively.

The multi-channel lens may include first and second multi-channel lenses respectively corresponding to the first and second lens frames, and the first and second multi-channel lenses may be configured to pass the image display light refracted by the first and second multi-channel lenses through different emission paths along the plurality of channels and to transfer the image display light in a user's eyeball disposition direction through a panel of the different emission paths.

Each of the first and second lens frames may include: a lens sheet having an area and a shape corresponding to the image display surface of the image display module; and a plurality of optical lenses on a front surface of the lens sheet and configured to refract the image display light passing through the lens sheet in an outer direction or an outer circumferential direction of the lens sheet and to emit the refracted image display light.

According to an embodiment of the present disclosure, a wearable device includes: a main frame supporting a transparent lens; an image display module configured to display an augmented reality content through the transparent lens; and an eyeball protection module on the main frame and configured to spray air, water, or tear solution to user's left and right eyes.

The image display module may include: an image display device assembled to at least one side of the main frame or formed integrally with the main frame and configured to display an augmented reality content image; and an image transmission member configured to transmit the augmented reality content image to the transparent lens, and the augmented reality content image may be displayed through reflective members formed in the image transmission member and the transparent lens.

The image display device may include: a partition wall partitioned in a matrix structure on a substrate; a plurality of light emitting elements, each in a plurality of emission areas arranged in the matrix structure partitioned by the partition wall and extending in a thickness direction of the substrate; base resins in the plurality of emission areas including the plurality of light emitting elements; and a plurality of optical patterns selectively on at least one of the plurality of emission areas.

The plurality of emission areas may include first to third emission areas or first to fourth emission areas in the matrix structure in each pixel area.

The eyeball protection module may include: first and second blink sensing units configured to sense blink motions of the user's left and right eyes in real time and to generate and output blink sensing signals of the user's left and right eyes; first and second temperature detection units configured to sense temperatures of the user's left and right eyes in real time and to output temperature sensing signals of the user's left and right eyes; first and second spray driving units configured to spray the air, water, or tear solution to the user's left and right eyes to induce user's blink motions; and a blink control unit configured to control spray driving of the first and second spray driving units based on at least one of the blink sensing signals of the user's left and right eyes and the temperature sensing signals for the user's left and right eyes.

The first and second blink sensing unit may include: an image sensor configured to capture images of the user's left and right eyes in real time; an image analyzer configured to compare captured eyeball images with each other in units of at least one frame and to detect eyelid images according to a comparison result; and a blink sensing signal output unit configured to generate blink sensing signals according to the detection of the eyelid images and to supply the blink sensing signals to the blink control unit.

The first and second temperature detection units may include: a contactless thermometer configured to sense the temperatures of the user's left and right eyes in real time; and an analog-to-digital converter configured to transmit the temperature sensing signals corresponding to the sensed temperatures of the user's left and right eyes to the blink control unit.

The first and second spray driving units may include: a storage capsule for storing the air, water, or tear solution; and a spray pump configured to spray the air, water, or tear solution to the user's left and right eyes during a period in which a spray control signal is input from the blink control unit.

A wearable device including an image display module, according to an embodiment of the present disclosure, may induce a user to blink to protect the user's eyes by monitoring a user's blink period and eyeball temperature and spraying air, moisture, a tear solution, or the like into the user's eyes.

In addition, the wearable device including an image display module, according to an embodiment of the present disclosure, may improve the user's satisfaction and reliability by displaying a fatigue increase message as an image according to a change in the user's blink period and eyeball temperature.

The aspects and features of the present disclosure are not limited to the aforementioned aspects and features, and various other aspects and features are included in the present disclosure.

### BRIEF DESCRIPTION OF THE DRAWINGS

The above and other aspects and features of the present disclosure will become more apparent by describing, in detail, embodiments thereof with reference to the attached drawings, in which:

FIG. 1 is an exploded perspective view of a wearable device including an image display module according to an embodiment of the present disclosure;

FIG. 2 is a diagram illustrating an internal configuration, in a face wearing direction, of a main frame illustrated in FIG. 1;

FIG. 3 is a front view illustrating a display panel illustrated in FIG. 1 and lens frames disposed on a front surface of the display panel;

FIG. 4 is a cross-sectional view taken along the line I-I′ of the lens frame illustrated in FIG. 3;

FIG. 5 is a front view illustrating multi-channel lenses disposed on front surfaces of the display panel and the lens frames illustrated in FIG. 1;

FIGS. 6(a) and 6(b) are perspective views illustrating, in detail, one side surface and another side surface of the multi-channel lens illustrated in FIGS. 1 and 5;

FIG. 7 is a front view illustrating, in detail, mirror coated areas of the multi-channel lens illustrated in FIGS. 6(a) and 6(b);

FIG. 8 is a rear perspective view illustrating, in detail, the mirror coated areas of the multi-channel lens illustrated in FIGS. 6(a) and 6(b);

FIG. 9 is an exploded perspective view illustrating an arrangement and a coupling structure of the display panel, the lens frame, and the multi-channel lens illustrated in FIGS. 1 and 5;

FIG. 10 is a cross-sectional view taken along the line A-A′ of the display device illustrated in FIG. 5;

FIG. 11 is a diagram illustrating an image displayed on the display panel when a user's pupil is positioned at the center;

FIG. 12 is a diagram illustrating a virtual reality (VR) image recognized by a user when the user's pupil is positioned at the center;

FIG. 13 is a block diagram illustrating an eyeball protection module configured to perform an eyeball protection function;

FIG. 14 is a flowchart sequentially describing eyeball protection operations of the eyeball protection module illustrated in FIG. 13;

FIG. 15 is an perspective view illustrating a wearable device including an image display module according to another embodiment of the present disclosure;

FIG. 16 is an exploded perspective view, in a rear direction, of an augmented reality providing device illustrated in FIG. 15;

FIG. 17 is an exploded perspective view, in a front direction, of the augmented reality providing device illustrated in FIG. 15;

FIG. 18 is a schematic exploded perspective view illustrating the image display module illustrated in FIGS. 15 to 17;

FIG. 19 is a layout diagram illustrating an image display device illustrated in FIG. 18;

FIG. 20 is a layout diagram illustrating the area A of FIG. 19 in detail;

FIG. 21 is a layout diagram illustrating pixels illustrated in the area B of FIG. 20;

FIG. 22 is a cross-sectional view illustrating an example of the image display device taken along the line I-I′ of FIG. 21; and

FIG. 23 is an enlarged cross-sectional view illustrating an example of a light emitting element of FIG. 22.

### DETAILED DESCRIPTION

The present disclosure will now be described more fully hereinafter with reference to the accompanying drawings, in which embodiments of the present disclosure are shown. This disclosure may, however, be embodied in different forms and should not be construed as limited to the embodiments set forth herein. Rather, these embodiments are provided so that this disclosure will be thorough and complete, and will fully convey the scope of the present disclosure to those skilled in the art.

It will be understood that when an element or layer is referred to as being “on,” “connected to,” or “coupled to” another element or layer, it may be directly on, connected, or coupled to the other element or layer or one or more intervening elements or layers may also be present. When an element or layer is referred to as being “directly on,” “directly connected to,” or “directly coupled to” another element or layer, there are no intervening elements or layers present. For example, when a first element is described as being “coupled” or “connected” to a second element, the first element may be directly coupled or connected to the second element or the first element may be indirectly coupled or connected to the second element via one or more intervening elements.

In the figures, dimensions of the various elements, layers, etc. may be exaggerated for clarity of illustration. The same reference numerals designate the same elements. As used herein, the term “and/or” includes any and all combinations of one or more of the associated listed items. Further, the use of “may” when describing embodiments of the present disclosure relates to “one or more embodiments of the present disclosure.” Expressions, such as “at least one of” and “any one of,” when preceding a list of elements, modify the entire list of elements and do not modify the individual elements of the list. For example, the expression “at least one of a, b, or c” indicates only a, only b, only c, both a and b, both a and c, both b and c, all of a, b, and c, or variations thereof. As used herein, the terms “use,” “using,” and “used” may be considered synonymous with the terms “utilize,” “utilizing,” and “utilized,” respectively. As used herein, the terms “substantially,” “about,” and similar terms are used as terms of approximation and not as terms of degree, and are intended to account for the inherent variations in measured or calculated values that would be recognized by those of ordinary skill in the art.

It will be understood that, although the terms first, second, third, etc. may be used herein to describe various elements, components, regions, layers, and/or sections, these elements, components, regions, layers, and/or sections should not be limited by these terms. These terms are used to distinguish one element, component, region, layer, or section from another element, component, region, layer, or section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

The terminology used herein is for the purpose of describing embodiments of the present disclosure and is not intended to be limiting of the present disclosure. As used herein, the singular forms “a” and “an” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components but do not preclude the presence or addition of one or more other features, integers, steps, operations, elements, components, and/or groups thereof.

Also, any numerical range disclosed and/or recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. § 112(a) and 35 U.S.C. § 132(a).

Each of the features of the various embodiments of the present disclosure may be combined or combined with each other, in part or in whole, and technically various interlocking and driving are possible. Each embodiment may be implemented independently of each other or may be implemented together in an association.

Hereinafter, embodiments will be described with reference to the accompanying drawings.

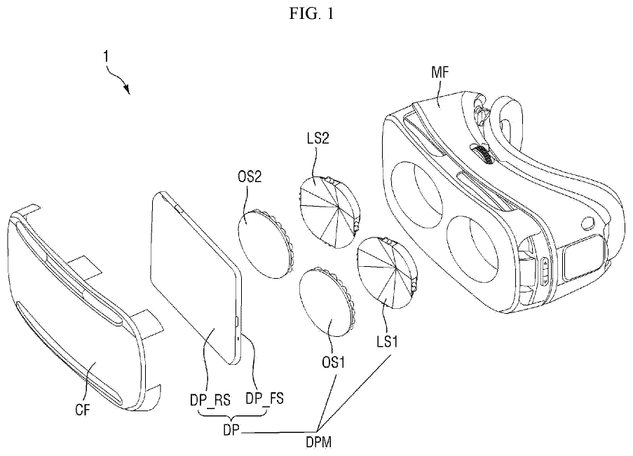

FIG. 1 is an exploded perspective view of a wearable device including an image display module according to an embodiment of the present disclosure.

An image display module DPM, according to an embodiment, may be formed integrally with a wearable device 1 that may be carried by a user and easily mounted on or detached from (e.g., removed from) a user's face or head. The image display module DPM may be formed in a shape in which it is assembled to the wearable device 1. The wearable device 1 may be formed in the shape of glasses or a head mount shape (e.g., a shape to be mountable to a user's head) and may provide an image to the user by using (or through) the image display module DPM. The wearable device 1 will be described as a see-closed type head mounted display device, but the present disclosure is not limited thereto. Other embodiments may include a see-through type head mounted display device.

Referring to FIG. 1, the wearable device 1 may include a main frame MF to be mounted on a user's body, the image display module DPM mounted on the main frame MF and to display an image, and a cover frame CF covering the image display module DPM.

The image display module DPM includes a display panel DP for displaying an image, first and second lens frames OS1 and OS2 for refracting image display light (e.g., light emitted from the display panel DP), and first and second multi-channel lenses LS1 and LS2 for forming light paths so that the image display light of the display panel DP is visible (e.g., is clearly visible) to the user.

The main frame MF may be worn on the user's face and head. The main frame MF may be formed in a shape corresponding to a shape and structure of the user's head and face.

The image display module DPM, that is, the display panel DP, the first and second lens frames OS1 and OS2, and the first and second multi-channel lenses LS1 and LS2, may be formed integrally with the main frame MF. In another embodiment, the display panel DP, the first and second lens frames OS1 and OS2, and the first and second multi-channel lenses LS1 and LS2 may be assembled to and mounted on the main frame MF. The main frame MF may have a space or structure in which the display panel DP, the first and second lens frames OS1 and OS2, and the first and second multi-channel lenses LS1 and LS2 may be accommodated. The main frame MF may further include a component or structure, such as a strap or a band, for easy mounting and may further include a display control unit, an image processing unit, a lens accommodating unit, and the like.

The display panel DP may be divided into (or may have or include) a front surface DP_FS, on which the image is displayed, and a rear surface DP_RS opposite to the front surface DP_FS. The image display light may be emitted to (or from) the front surface DP_FS of the display panel DP. As described later, the first and second lens frames OS1 and OS2 may be disposed on (or aligned with) the front surface DP_FS of the display panel DP, and the first and second multi-channel lenses LS1 and LS2 may be disposed on (or aligned with) front surfaces of the first and second lens frames OS1 and OS2, respectively.

The display panel DP may be embedded in the main frame MF or may be detachably assembled to the main frame MF in a state in which the first and second lens frames OS1 and OS2 and the first and second multi-channel lenses LS1 and LS2 are mounted thereon and fixed thereto. The display panel DP may be configured to be opaque, transparent, or translucent according to a design of the image display module DPM, for example, according to an intended use of the image display module DPM.

The display panel DP may be a light emitting display panel including light emitting elements. For example, the display panel DP may be an organic light emitting display panel including (or using) an organic light emitting diode, a micro light emitting diode display panel including (or using) a micro light emitting diode (LED), a quantum dot light emitting display panel including (or using) a quantum dot light emitting diode, or an inorganic light emitting display panel including (or using) an inorganic light emitting element. Hereinafter, the display panel DP will be described as being a micro light emitting diode display panel as an example, but the present disclosure is not limited thereto.

Each of the first and second lens frames OS1 and OS2 may have an area corresponding to an image display surface of the display panel DP and may be formed in a shape corresponding to the image display surface. In addition, the first and second lens frames OS1 and OS2 may be formed (or arranged) in areas and shapes corresponding to shapes of rear surfaces of the first and second multi-channel lenses LS1 and LS2, respectively. Rear surfaces of the first and second lens frames OS1 and OS2 are attached to the image display surface of the display panel DP, and the first and second multi-channel lenses LS1 and LS2 are attached to the front surfaces of the first and second lens frames OS1 and OS2, respectively. The first and second lens frames OS1 and OS2 refract the image display light emitted from the image display surface of the display panel DP at an angle (e.g., a preset angle) and provide the refracted image display light to the first and second multi-channel lenses LS1 and LS2 disposed on the front surfaces thereof, respectively.

For example, the first and second lens frames OS1 and OS2 refract the image display light emitted in a front direction from the image display surface of the display panel DP in an outer direction (or an outer circumferential direction) as compared with (e.g., relative to) the front direction and provide the refracted image display light to the first and second multi-channel lenses LS1 and LS2 disposed on the front surfaces thereof, respectively. The first and second lens frames OS1 and OS2 may refract the image display light incident on the rear surface thereof in the outer direction (or the outer circumferential direction) and may provide the refracted image display light to the rear surfaces of the first and second multi-channel lenses LS1 and LS2, respectively. A refraction angle or a radiation angle at which the image display light is refracted in the outer direction of each of the first and second lens frames OS1 and OS2 may be in a range of about 1° to about 30°. A detailed structure and the light refraction angle of each of the first and second lens frames OS1 and OS2 will be described, in detail, later with reference to the accompanying drawings.

The first and second multi-channel lenses LS1 and LS2 form paths of the light emitted through the first and second lens frames OS1 and OS2 to allow the image display light to be visible to user's eyes in the front direction.

The first and second multi-channel lenses LS1 and LS2 may provide a plurality of channels (or paths) through which the image display light emitted from the display panel DP passes, respectively. The plurality of channels may pass the image display light emitted from the display panel DP through different paths and may provide the image display light to the user. The image display light emitted through the first and second lens frames OS1 and OS2 may be incident on the respective channels, and images magnified through the respective channels may be focused on the user's eyes.

The first and second multi-channel lenses LS1 and LS2 may be arranged on the front surfaces of the first and second lens frames OS1 and OS2 to correspond to positions of user's left and right eyes, respectively. The first and second multi-channel lenses LS1 and LS2 may be accommodated inside the main frame MF.

The first and second multi-channel lenses LS1 and LS2 refract and reflect the image display light emitted through the first and second lens frames OS1 and OS2 at least once to form paths to the user's eyes. At least one infrared light source may be disposed on one side of each of the first and second multi-channel lenses LS1 and LS2 facing the main frame MF or the user's eyeballs.

The cover frame CF may be disposed in the rear surface DP_RS direction (e.g., may be arranged over or on the rear surface DP_RS) of the display panel DP to cover and to protect the display panel DP. The cover frame CF may cover the display panel DP and may be mounted on the main frame MF.

The image display module DPM may further include a display control unit for controlling overall operations of the image display module DPM including the display panel DP. The display control unit may control an image display operation, an audio device, and the like, of the display panel DP. The display control unit may be mounted on the display panel DP or the main frame MF or may be embedded in the main frame MF and electrically connected to the display panel DP. Configuration features and functional features of the display control unit will be described, in detail, later with reference to the accompanying drawings.

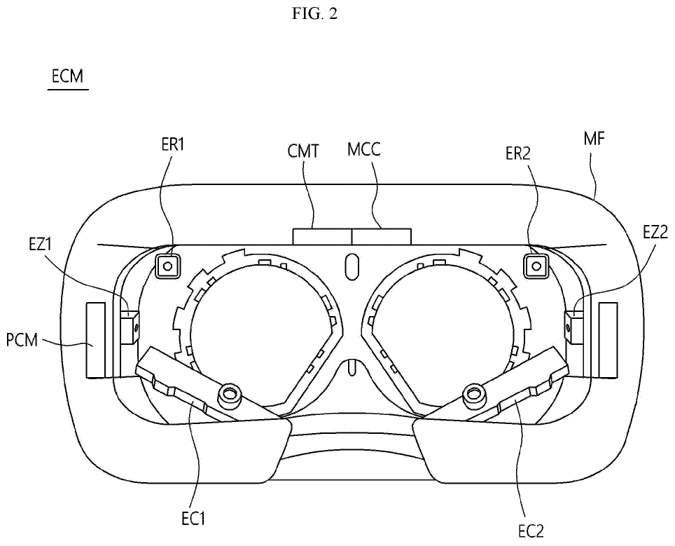

FIG. 2 is a diagram illustrating an internal configuration, in a face wearing direction, of the main frame illustrated in FIG. 1.

Referring to FIG. 2, an eyeball protection module ECM, which protects the user's eyeballs, is disposed in a rear direction or a wearing direction of the main frame MF, in other words, in a direction in which the main frame MF is worn on the user's face or eye areas. In addition, a display control unit PCM for controlling overall operations of the image display module DPM may be disposed inside the main frame MF.

The eyeball protection module ECM includes first and second blink sensing units EC1 and EC2, first and second temperature detection units ER1 and ER2, a use time detection unit CMT, first and second spray driving units EZ1 and EZ2, and a blink control unit MCC.

The first and second blink sensing units EC1 and EC2 are configured to sense blink motions of the user's left and right eyes in real time and to generate and output blink sensing signals of the user's left and right eyes. To this end, each of the first and second blink sensing units EC1 and EC2 may include an image sensor, an image analyzer, and a blink sensing signal output unit.

The first and second temperature detection units ER1 and ER2 are configured to sense temperatures of the user's left and right eyes in real time and to output temperature sensing signals of the user's left and right eyes, respectively. The first and second temperature detection units ER1 and ER2 may include at least one thermometer, a signal conversion circuit, and the like.

The use time detection unit CMT is configured to monitor an image display period of the display panel DP and to accumulate and output information on a gaze period during which the user gazes at a displayed image. In addition, the use time detection unit CMT is configured to transmit the accumulated information on the gaze period to the blink control unit MCC in units of a period (e.g., a preset or predetermined period).

The first and second spray driving units EZ1 and EZ2 are configured to spray at least one of air, water, and a tear solution to the user's left and right eyes to induce user's blink motions. To this end, the blink control unit MCC is configured to control spray driving of the first and second spray driving units EZ1 and EZ2 based on at least one signal or information of the blink sensing signals of the user's left and right eyes, the temperature sensing signals for the user's left and right eyes, and the information on the gaze period during which the user gazes at the displayed image.

The blink control unit MCC may be formed in a one-chip type with the display control unit PCM; that is, the blink control unit MCC may be integrated with the display control unit PCM.

The display control unit PCM may perform image processing (e.g., image mapping) according to image display paths and a magnification according to the first and second lens frames OS1 and OS2 and the first and second multi-channel lenses LS1 and LS2 and may control the display panel DP to display the mapped image. The display control unit PCM may be implemented as a dedicated processor including an embedded processor or the like and/or a general-purpose processor including a central processing unit or an application processor but is not limited thereto.

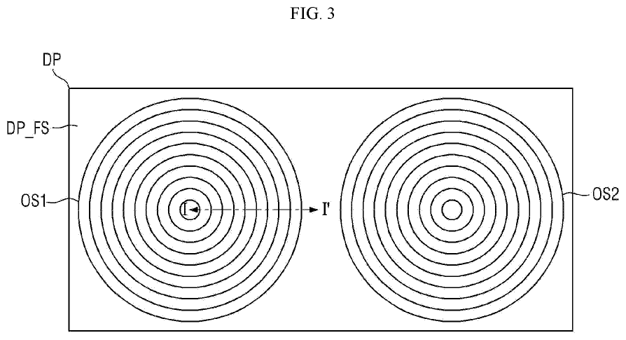

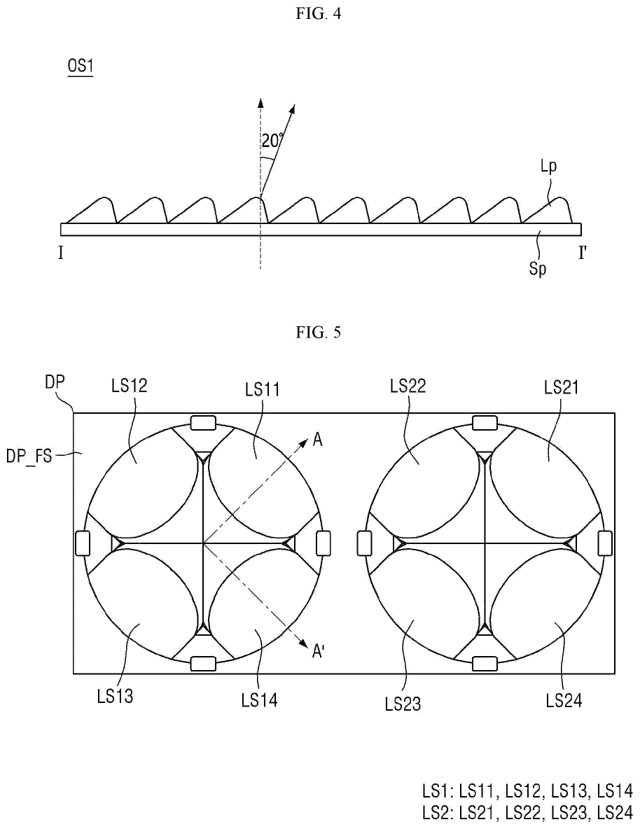

FIG. 3 is a front view illustrating a display panel and lens frames disposed on a front surface of the display panel illustrated in FIG. 1, and FIG. 4 is a cross-sectional view of the lens frame taken along the line I-I′ in FIG. 3.

Referring to FIGS. 3 and 4, each of the first and second lens frames OS1 and OS2 includes a lens sheet Sp and a plurality of optical lenses Lp.

The lens sheet Sp may have an area corresponding to the image display surface of the display panel DP and may be formed in a shape corresponding to the image display surface. The lens sheet Sp may be formed of a flat transparent film and may have a refractive index (e.g., a predetermined refractive index) set in advance according to a material of the film. Such a lens sheet Sp may be formed in a size and a shape configured to (e.g., sufficient to) cover the image display surface of the display panel DP. An adhesive material may be formed on at least one of a front surface and a rear surface of the lens sheet Sp.

The plurality of optical lenses Lp are disposed on the front surface of the lens sheet Sp, refract the image display light passing through the lens sheet Sp in an outer direction (or an outer circumferential direction) of the lens sheet Sp, and emit the refracted image display light. The plurality of optical lenses Lp may be each formed in ring types having different circumferential lengths (e.g., having different circumferences) and may be arranged as a plurality of concentric circles on the front surface of the lens sheet Sp to cover the entirety of the front surface of the lens sheet Sp. At least one structural feature, such as a length, a formation area, a width in at least one direction, and a breadth, of each of the optical lenses Lp in the ring types may be different between adjacent optical lenses Lp. Each of the plurality of optical lenses Lp may have a hemispherical cross-sectional shape or a convex protrusion cross-sectional shape to radially radiate the image display light passing through the lens sheet Sp on a rear surface thereof and incident thereon to a front surface thereof.

Referring to FIG. 4, each of the plurality of optical lenses Lp may be formed so that the highest point (or the thickest point) of the cross-section is disposed at a position inclined at a first angle (e.g., about 20° in FIG. 4) from a front vertical direction (the dotted line arrow in FIG. 4) of each optical lens Lp to the outer direction. In other words, the thickest point of the cross section of each of the plurality of optical lenses Lp may be disposed at a point (e.g., may have a surface or a point surface) inclined at a first angle (e.g., a preset or predetermined first angle) toward the outer circumferential direction of the lens sheet Sp based on the front vertical direction (e.g., the dotted line arrow) of each optical lens Lp. Each optical lens Lp may refract the image display light at the first angle in the outer direction of the lens sheet Sp according to such a cross-sectional shape and inclination angle of each optical lens Lp and may emit the refracted image display light.

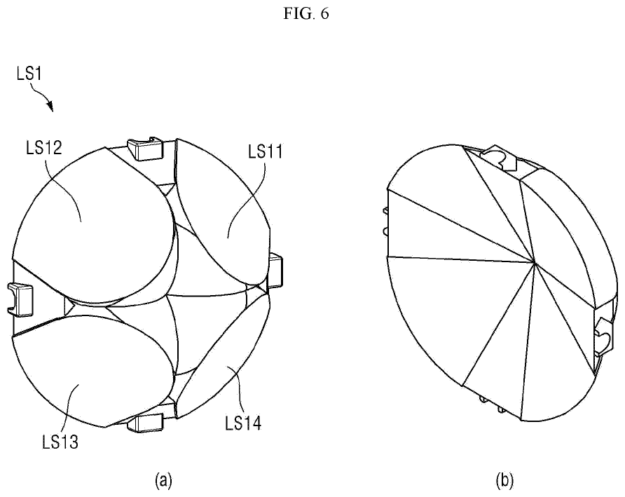

FIG. 5 is a front view illustrating multi-channel lenses disposed on front surfaces of the display panel and the lens frames illustrated in FIG. 1, and FIGS. 6(a) and 6(b) are perspective views illustrating one side surface and another side surface of the multi-channel lens illustrated in FIGS. 1 and 5.

Referring to FIGS. 5 to 6(b), the first and second multi-channel lenses LS1 and LS2 may be disposed on the front surfaces the first and second lens frames OS1 and OS2, respectively, and may be positioned at points corresponding to the user's left and right eyes, respectively. For example, the display panel DP may have an approximately rectangular shape long (or elongated) in left and right directions (e.g., the horizontal direction in FIG. 5) in a plan view. The first multi-channel lens LS1 may be disposed on the first lens frame OS1 on the front surface of one side of the display panel DP, that is, on the front surface of the first lens frame OS1, and the second multi-channel lens LS2 may be disposed on the second lens frame OS2 on the front surface of the other side of the display panel DP, that is, on the front surface of the second lens frame OS2.

In an embodiment, the first and second multi-channel lenses LS1 and LS2 may be disposed to be symmetrical to each other with respect to the center of the display panel DP and may have substantially the same or similar structure but are not limited thereto.

The first and second multi-channel lenses LS1 and LS2 may include a plurality of sub-lenses LS11, LS12, LS13, LS14, LS21, LS22, LS23, and LS24.

In an embodiment, the first multi-channel lens LS1 may include a first sub-lens LS11, a second sub-lens LS12, a third sub-lens LS13, and a fourth sub-lens LS14. The second multi-channel lens LS2 may include a fifth sub-lens LS21, a sixth sub-lens LS22, a seventh sub-lens LS23, and an eighth sub-lens LS24. However, the number of sub-lenses LS11, LS12, LS13, LS14, LS21, LS22, LS23, and LS24 is not limited thereto.

In an embodiment, the second multi-channel lens LS2 is substantially the same as or similar to the first multi-channel lens LS1, and thus, the first multi-channel lens LS1 will hereinafter be primarily described.

The first multi-channel lens LS1 illustrated in FIG. 5 may have a substantially circular shape in a plan view. The first sub-lens LS11, the second sub-lens LS12, the third sub-lens LS13, and the fourth sub-lens LS14 may be disposed in, for example, a clover shape surrounding (or arranged around) the center of the circular shape in a plan view. For example, as illustrated in FIG. 5, the first sub-lens LS11, the second sub-lens LS12, the third sub-lens LS13, and the fourth sub-lens LS14 may be disposed on the upper right side, the upper left side, the lower left side, and the lower right side with respect to the center of the first multi-channel lens LS1, respectively. The first sub-lens LS11, the second sub-lens LS12, the third sub-lens LS13, and the fourth sub-lens LS14 may be integrally connected to each other or may be separated from each other.

FIG. 6(a) is a perspective view illustrating one side (e.g., a convex side) of the first multi-channel lens LS1 facing the user's eye, and FIG. 6(b) is a perspective view illustrating another side (e.g., the opposite side or the concave side) of the first multi-channel lens LS1 facing the image display surface of the display panel DP.

Referring to FIGS. 6(a) and 6(b), a cross-section of the first multi-channel lens LS1 may be formed in an approximate hemispherical shape. In such an embodiment, one side of the first multi-channel lens LS1 facing the main frame MF or the user's eye may be formed in a convex shape, and the other side of the first multi-channel lens LS1 facing the display panel DP may be formed in a concave shape.

A cross-section of the second multi-channel lens LS2 may also be formed in an approximate hemispherical shape, and the fifth sub-lens LS21, the sixth sub-lens LS22, the seventh sub-lens LS23, and the eighth sub-lens LS24 may be disposed in a circular shape or a clover shape surrounding the center of the second multi-channel lens LS2 in a plan view.

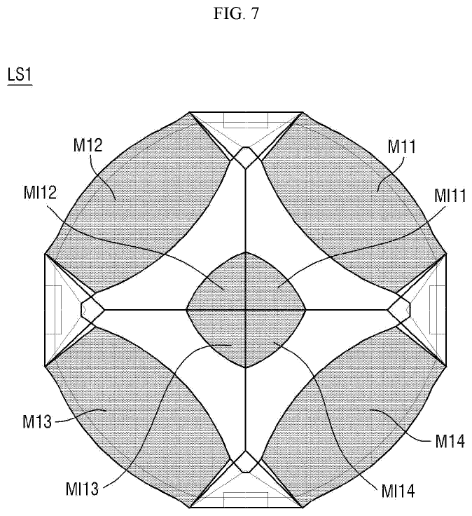

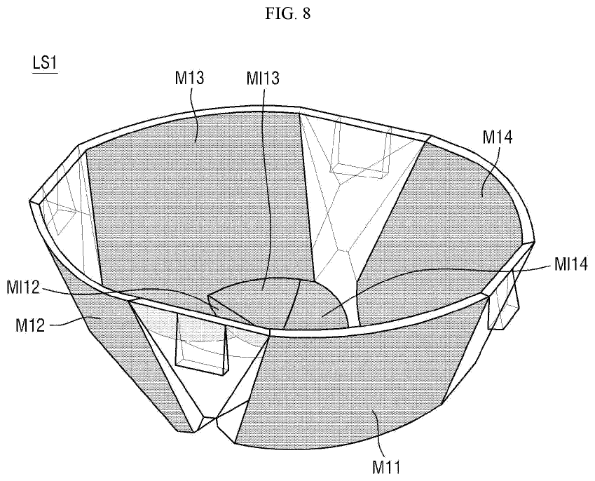

FIG. 7 is a front view illustrating mirror coated areas of the multi-channel lens illustrated in FIGS. 6(a) and 6(b), and FIG. 8 is a rear perspective view illustrating the mirror coated areas of the multi-channel lens illustrated in FIGS. 6(a) and 6(b).

Referring to FIGS. 7 and 8, front or rear surfaces of the first to fourth sub-lenses LS11, LS12, LS13, and LS14 formed in the first multi-channel lens LS1 may be mirror coated areas. Accordingly, a reflective material may be formed or coated on first to fourth mirror coated areas M11, M12, M13, and M14 divided respectively in the first to fourth sub-lenses LS11, LS12, LS13, and LS14.

The first to fourth mirror coated areas M11, M12, M13, and M14 divided respectively in the first to fourth sub-lenses LS11, LS12, LS13, and LS14 face a concave shape portion of the first multi-channel lens LS1, which is a central portion of the first multi-channel lens LS1. Accordingly, the first to fourth mirror coated areas M11, M12, M13, and M14 may reflect the image display light incident from the rear surface of the first multi-channel lens LS1 toward the concave shape portion, which is the central portion of the first multi-channel lens LS1.

In addition, first to fourth inner coated areas MI11, MI12, MI13, and MI14 facing the first to fourth mirror coated areas M11, M12, M13, and M14 are defined in the concave shape portion, which is the central portion of the first multi-channel lens LS1 and the rear surface of the first multi-channel lens LS1. A reflective material is formed or coated on the first to fourth inner coated areas MI11, MI12, MI13, and MI14 in the same manner as the first to fourth mirror coated areas M11, M12, M13, and M14. Accordingly, the first to fourth inner coated areas MI11, MI12, MI13, and MI14 may reflect the image display light reflected from the first to fourth mirror coated areas M11, M12, M13, and M14 in a direction toward the user's eyeball, which is the front direction.

Formation structures of the first to fourth mirror coated areas M11, M12, M13, and M14 and the first to fourth inner coated areas MI11, MI12, MI13, and MI14 of the first multi-channel lens LS1 are equally applied to the channel lens LS2.

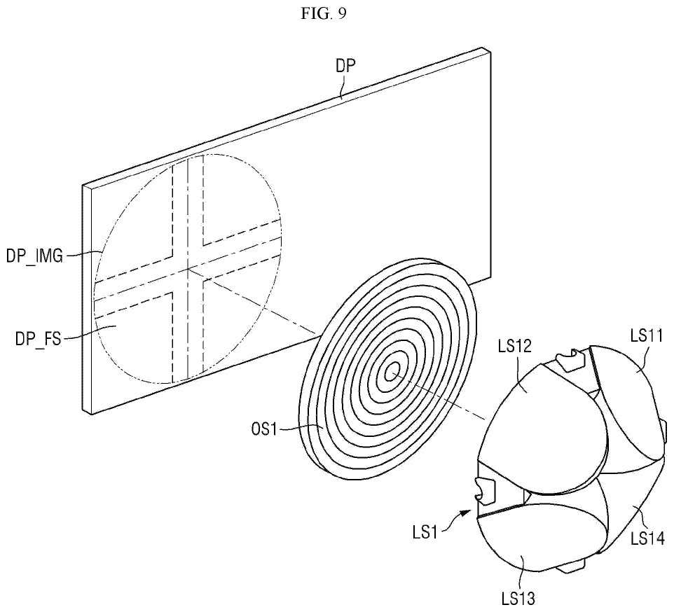

FIG. 9 is an exploded perspective view illustrating an arrangement and a coupling structure of the display panel, the lens frame, and the multi-channel lens illustrated in FIGS. 1 and 5, and FIG. 10 is a cross-sectional view of the display device taken along the line A-A′ illustrated in FIG. 5.

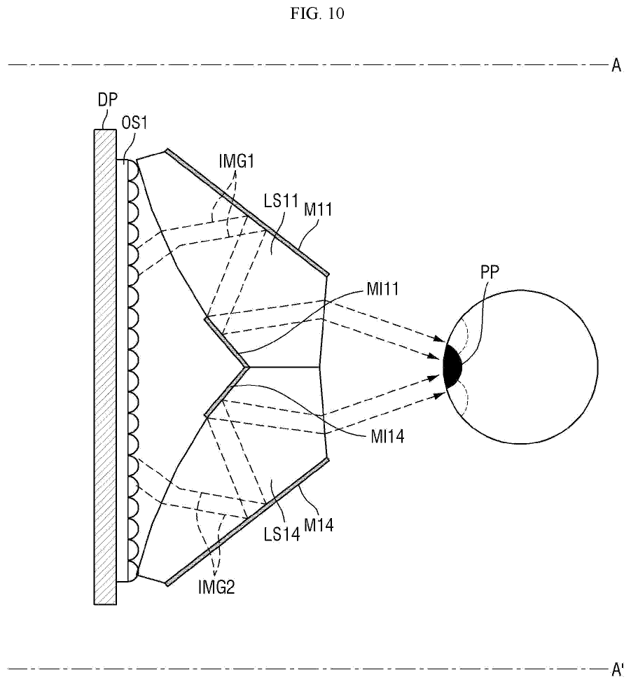

Referring to FIGS. 9 and 10, the first and second lens frames OS1 and OS2 may be attached to left eye and right eye image display surfaces of the display panel DP, respectively, and the first and second multi-channel lenses LS1 and LS2 may be attached to the front surfaces of the first and second lens frames OS1 and OS2, respectively.

When the user frontally gazes at a display image DP_IMG and/or a VR image, to be described later, through the first and second multi-channel lenses LS1 and LS2, the display panel DP displays the display image DP_IMG corresponding to a front pupil PP direction of the user on the left and right eye image display surfaces.

For example, the plurality of sub-lenses LS11, LS12, LS13, LS14, LS21, LS22, LS23, and LS24 formed in the first and second multi-channel lenses LS1 and LS2 provides, respectively, a plurality of channels through which the light emitted from the front surface DP_FS of the display panel DP passes. The image display light emitted from different areas of the front surface DP_FS of the display panel DP may pass through different channels through different paths. Here, each image display light may include a partial image for constituting one complete VR image.

For example, as illustrated in FIG. 10, the first sub-lens LS11 provides a channel through which image display light IMG1 emitted from one area of the display panel DP (e.g., an upper end of the display panel DP) passes, and the fourth sub-lens LS14 may provide a channel through which image display light IMG2 emitted from the other area of the display panel DP (e.g., a lower end of the display panel DP) passes. One area and the other area of the display panel DP may include an area overlapping the first sub-lens LS11 and an area overlapping the fourth sub-lens LS14, respectively.

Likewise, the second sub-lens LS12 and the third sub-lens LS13 may provide channels through which light emitted from different areas of the display panel DP passes.

In an embodiment, the image display light passing through each of the sub-lenses LS11, LS12, LS13, LS14, LS21, LS22, LS23, and LS24 may be reflected twice, first by the first to fourth mirror coated areas M11, M12, M13, and M14 and then by the first to fourth inner coated areas MI11, MI12, MI13, and MI14 to be provided to the user but is not limited thereto.

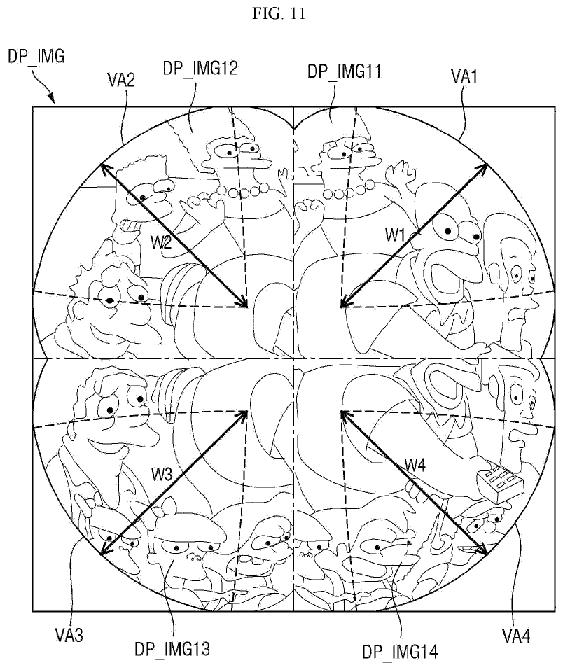

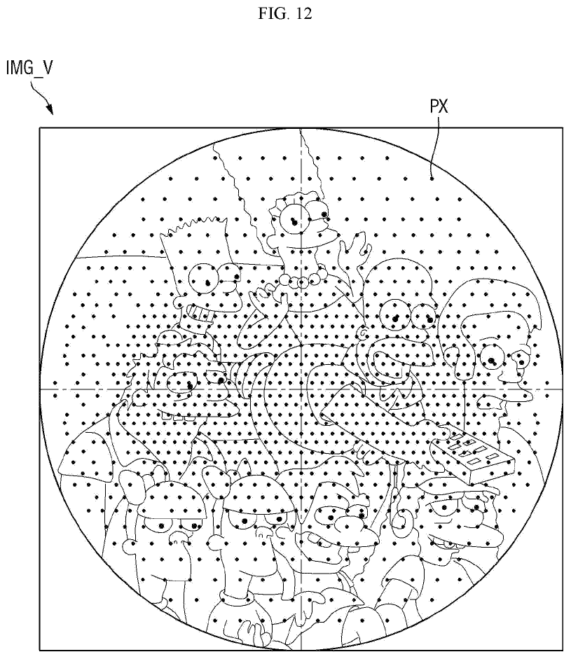

FIG. 11 is a diagram illustrating an image displayed on the display panel when a user's pupil is positioned at the center, and FIG. 12 is a diagram illustrating a virtual reality (VR) image recognized by a user when the user's pupil is positioned at the center.

Referring to FIG. 11, the display panel DP may display a display image DP_IMG divided into four display images DP_IMG. In a plan view, the display image DP_IMG may include a first divided display image DP_IMG11, a second divided display image DP_IMG12, a third divided display image DP_IMG13, and a fourth divide display image DP_IMG14 arranged in a counterclockwise direction based on the center of the display image DP_IMG when viewing the front surface DP_FS of the display panel DP.

When the user's pupil PP gazes approximately at a central portion, the first divided display image DP_IMG11, the second divided display image DP_IMG12, the third divided display image DP_IMG13, and the fourth divided display image DP_IMG14 may be displayed in (or may have) approximately the same size. Sizes of the divided display images DP_IMG may refer to widths of the divided display images DP_IMG in a radial direction (e.g., a diagonal direction) based on the center of the display image DP_IMG. However, the present disclosure is not limited thereto, and the sizes of the divided display images DP_IMG may also refer to widths of the divided display images DP_IMG in a horizontal direction and/or widths of the divided display images DP_IMG in a vertical direction in a plan view.

As illustrated in FIG. 11, the sizes of the first divided display image DP_IMG11, the second divided display image DP_IMG12, the third divided display image DP_IMG13, and the fourth divided display image DP_IMG14 may be measured based on boundaries of a first divided viewing area VA1, a second divided viewing area VA2, a third divided viewing area VA3, and a fourth divided viewing area VA4 but are not limited thereto. The sizes of the first divided display image DP_IMG11, the second divided display image DP_IMG12, the third divided display image DP_IMG13, and the fourth divided display image DP_IMG14 may be measured based on an intersection point of boundaries disposed between the first divided display image DP_IMG11, the second divided display image DP_IMG12, the third divided display image DP_IMG13, and the fourth divided display image DP_IMG14.

As illustrated in FIG. 11, a first width W1 of the first divided display image DP_IMG11, a second width W2 of the second divided display image DP_IMG12, a third width W3 of the third divided display image DP_IMG13, and a fourth width W4 of the fourth divided display image DP_IMG14 may be substantially the same as each other. Accordingly, the first divided display image DP_IMG11, the second divided display image DP_IMG12, the third divided display image DP_IMG13, and the fourth divided display image DP_IMG14 may be displayed on the display panel DP at substantially the same magnification.

Referring to FIG. 12, the display device 10 may output a foveated rendered VR image IMG_V to the display panel DP based on a position of the user's pupil PP. Here, foveated rendering is one of rendering technologies of improving image quality of a sight line gaze area while significantly reducing image quality of a peripheral visual field to reduce a rendering work amount (e.g., to reduce rendering workload). For example, the foveated rendering may refer to an image processing method of reducing or minimizing a graphic calculation load while implementing a high image quality VR experience with high immersion by displaying an area at which a user's sight line gazes with maximum image quality while expressing the other areas at relatively low image quality. In addition, the VR image IMG_V may refer to an image passing through each of the first and second multi-channel lenses LS1 and LS2 to be recognized by the user. Referring to FIGS. 11 and 12, the VR image IMG_V may be generated by a combination of portions of a plurality of divided display images DP_IMG11, DP_IMG12, DP_IMG13, and DP_IMG14.

The first divided display image DP_IMG11, the second divided display image DP_IMG12, the third divided display image DP_IMG13, and the fourth divided display image DP_IMG14 may include the first divided viewing area VA1, the second divided viewing area VA2, the third divided viewing area VA3, and the fourth divided viewing area VA4, respectively.

The first divided viewing area VA1, the second divided viewing area VA2, the third divided viewing area VA3, and the fourth divided viewing area VA4 may be defined by, for example, optical characteristics of the first and second multi-channel lenses LS1 and LS2, a user's gaze direction, and the like. Shapes, sizes, and/or magnifications of the first divided viewing area VA1, the second divided viewing area VA2, the third divided viewing area VA3, and the fourth divided viewing area VA4 may vary depending on the optical characteristics of the first and second multi-channel lenses LS1 and LS2, the user's gaze direction, and the like.

When the user's pupil PP is positioned approximately at a center point, the display panel DP may display the display image DP_IMG so that a magnification of a central area of the display image DP_IMG is greater than that of a peripheral area of the display image DP_IMG surrounding (or around) the central area.

As illustrated in FIG. 12, the central area of the VR image IMG_V may have a pixel PX density relatively higher than that of the peripheral area surrounding the central area. For example, the pixel PX density may increase from an edge of the VR image IMG_V toward the center of the VR image IMG_V. Accordingly, the central area of the VR image IMG_V may be displayed with higher image quality than the surrounding area.

The central area of the VR image IMG_V may refer to an intersection point of boundaries between an image of the first divided viewing area VA1, an image of the second divided viewing area VA2, an image of the third divided viewing area VA3, and an image of the fourth divided viewing area VA4 combined with each other and recognized by the user and an adjacent area surrounding the intersection point but is not limited thereto.

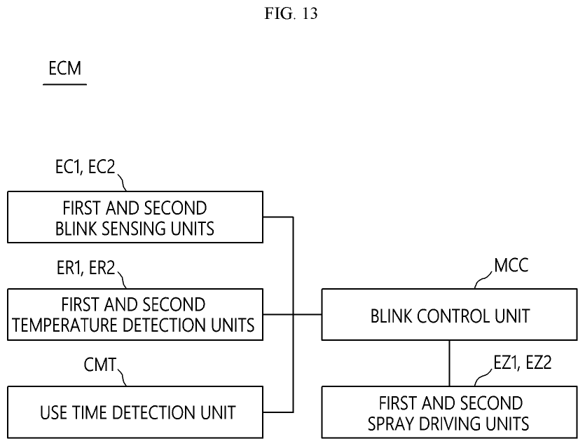

FIG. 13 is a block diagram describing an eyeball protection module that performs an eyeball protection function.

As illustrated in FIG. 13, the eyeball protection module ECM includes first and second blink sensing units EC1 and EC2, first and second temperature detection units ER1 and ER2, a use time detection unit CMT, first and second spray driving units EZ1 and EZ2, and a blink control unit MCC.

The first and second blink sensing units EC1 and EC2 are configured to sense blink motions of the user's left and right eyes in real time and to generate and output blink sensing signals of the user's left and right eyes. To this end, each of the first and second blink sensing units EC1 and EC2 may include an image sensor, an image analyzer, and a blink sensing signal output unit. Each of the first and second blink sensing units EC1 and EC2 captures images of the user's left and right eyes in real time using the image sensor. Each of the first and second blink sensing units EC1 and EC2 compares captured eyeball images with each other in units of at least one frame and detects an eyelid image according to a comparison result by using the image analyzer. Subsequently, each of the first and second blink sensing units EC1 and EC2 outputs a blink sensing signal according to the detection of the eyelid image by using the blink sensing signal output unit. Blink sensing signals of the user's left and right eyes each output from the first and second blink sensing units EC1 and EC2 are transmitted to the blink control unit MCC in real time.

The first and second temperature detection units ER1 and ER2 are configured to sense temperatures of the user's left and right eyes, respectively, in real time and to output temperature sensing signals of the user's left and right eyes, respectively. To this end, the first and second temperature detection units ER1 and ER2 may include contactless infrared thermometers (or infrared cameras), infrared radiation thermometers, or the like. The first and second temperature detection units ER1 and ER2 may sense the temperatures of the user's left and right eyes in real time using the respective contactless thermometers and may transmit the temperature sensing signals of the user's left eye and the right eye to the blink control unit MCC by using analog-to-digital (AD) converters, respectively.

The use time detection unit CMT is configured to monitor an image display period of the display panel DP and to accumulates and output information on a gaze period during which the user gazes at a displayed image. The use time detection unit CMT is configured to count a period during which image data or control signals from the display control unit PCM are transmitted to the display panel DP with a count circuit and to accumulate information on a period during which an image is displayed on the display panel DP, that is, the gaze period during which the user gazes at the displayed image. In addition, the use time detection unit CMT is configured to transmit the accumulated information on the gaze period to the blink control unit MCC in units of a period (e.g., a preset period).

The first and second spray driving units EZ1 and EZ2 are configured to spray at least one of air, water, and a tear solution to the user's left and right eyes to induce user's blink motions. For example, the first and second spray driving units EZ1 and EZ2 may spray at least one of air, water, and the tear solution to the user's left and right eyes, respectively, by using a storage capsule of (or containing) at least one of air, water, and the tear solution and a spray pump during a period in which spray control signals are input from the blink control unit MCC.

The blink control unit MCC is configured to control spray driving of the first and second spray driving units EZ1 and EZ2 based on at least one signal or information of the blink sensing signals of the user's left and right eyes, the temperature sensing signals for the user's left and right eyes, and the information on the gaze period during which the user gazes at the displayed image. For example, the blink control unit MCC is configured to detect the number of inputs of the blink sensing signals of the user's left and right eyes, that is, the number of blinks, in units of a reference time (e.g., over a certain amount of time or time period). In addition, the blink control unit MCC is configured to compare the number of inputs of the blink sensing signals for each reference time unit with a reference value (e.g., a reference number of times) for each reference time.

The blink control unit MCC is configured to transmit spray control signals to the first and second spray driving units EZ1 and EZ2 when the number of inputs of the blink sensing signals for each reference time unit is detected (or determined) to be below (or less than or smaller than) the reference value for each reference time. In addition, the blink control unit MCC is configured to transmit an eyeball protection operation execution notification message to the display control unit PCM to allow the eyeball protection operation execution notification message to be displayed on the display panel DP under the control of the display control unit PCM when the number of inputs of the blink sensing signals for each reference time unit is detected to be smaller than the reference value for each reference time unit.

In addition, the blink control unit MCC is configured to receive the temperature sensing signals of the user's left and right eyes in real time through the first and second temperature detection units ER1 and ER2 and to detect temperature values of the user's left and right eyes corresponding to the temperature sensing signals of the user's left and right eyes using a memory or a register. In addition, the blink control unit MCC is configured to compare the temperature values of the user's left and right eyes with a threshold temperature value (e.g., a preset threshold temperature value).

The blink control unit MCC is configured to transmit spray control signals to the first and second spray driving units EZ1 and EZ2 when the temperature values of the user's left and right eyes are (or become) greater than the threshold temperature value. In addition, the blink control unit MCC may transmit an eyeball protection operation execution notification message to the display control unit PCM.

The blink control unit MCC is configured to receive the accumulated information on the gaze period during which the user gazes at the displayed image in real time from the use time detection unit CMT. In addition, the blink control unit MCC is configured to compare the information on the gaze period of the user with use time reference information for each step (or period).

The blink control unit MCC may transmit spray control signals to the first and second spray driving unit EZ1 and EZ2 whenever the information on the gaze period of the user becomes the same as the use time reference information for each step. In addition, the blink control unit MCC may transmit an eyeball protection operation execution notification message to the display control unit PCM.

When the eyeball protection operation execution notification message is received from the blink control unit MCC, the display control unit PCM corrects image data by including the eyeball protection operation execution notification message or a fatigue increase message in the image data. In addition, the display control unit PCM supplies the corrected image data to the display panel DP to allow the eyeball protection operation execution notification message or the fatigue increase message to be displayed on the display panel DP.

When the eyeball protection operation execution notification message is received from the blink control unit MCC, the display control unit PCM is configured to generate luminance correction image data of n frames by further increasing luminance values of the image data by a correction luminance value (e.g., a preset correction luminance value) during an n-frame period (e.g., a preset n-frame period). Here, n is a positive integer. As such, the display control unit PCM may improve the user's visibility by supplying the luminance correction image data of the n frames of which the luminance values are further increased as a whole by the correction luminance value to the display panel DP to allow an image of which the luminance values are increased to be displayed on the display panel DP. When the user blinks according to the spray driving of the first and second spray driving units EZ1 and EZ2, a user's eyesight may be blurred during the next n-frame period. Therefore, the display control unit PCM may increase the visibility of the user by allowing an image of which the luminance values are increased as a whole (or overall) by the correction luminance value to be displayed on the display panel DP.

When the eyeball protection operation execution notification message is received from the blink control unit MCC, the display control unit PCM may allow image data of a current frame to be equally delayed and displayed on the display panel DP for an n-frame period. For example, when the eyeball protection operation execution notification message is received, the display control unit PCM may equally copy image data of the current frame and supply the copied image data to the display panel DP for an n-frame period. Accordingly, the same current image data may be displayed on the display panel DP during the n-frame period. Even though the user blinks, a current image is delayed and displayed during a period in which the user blinks such that the user may continuously view the image without missing the image.

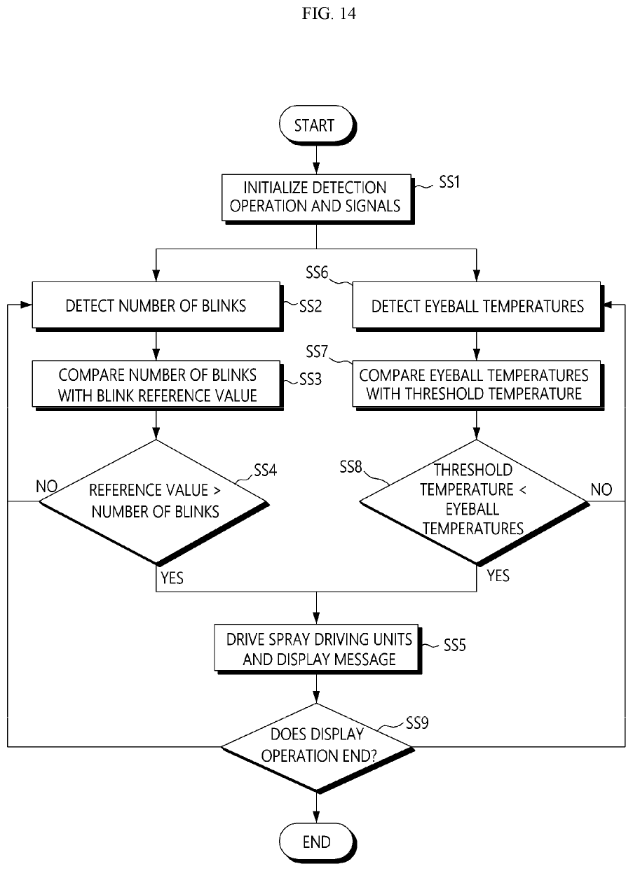

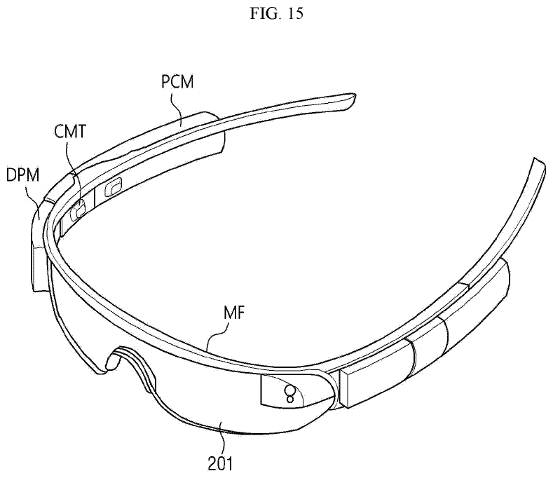

FIG. 14 is a flowchart describing eyeball protection operations of the eyeball protection module illustrated in FIG. 13.

Referring to FIG. 14, the use time detection unit CMT of the eyeball protection module ECM monitors an operation timing of the display panel DP and to transmit a start signal to the blink control unit MCC when the display panel DP operates. The use time detection unit CMT resets, initializes, and counts a use time of the display panel DP, and the blink control unit MCC initializes initial operation values of the first and second blink sensing units EC1 and EC2 and the first and second temperature detection unit ER1 and ER2 (SS1).

The first and second blink sensing units EC1 and EC2 sense blink motions of the user's left and right eyes in real time from a point in time initialized by the blink control unit MCC and generates and outputs blink sensing signals of the user's left and right eyes according to the blink motions of the user's left and right eyes (SS2). The blink sensing signals of the user's left and right eyes each output from the first and second blink sensing units EC1 and EC2 are transmitted to the blink control unit MCC.

The blink control unit MCC compares the number of inputs of the blink sensing signals with a reference value for each reference time. For example, the blink control unit MCC may compare the number of inputs of the blink sensing signals with a reference value for each reference time, such as one minute, two minutes, three minutes, four minutes, and five minutes (SS3).

When the number of inputs of the blink sensing signals for each reference time unit is detected to be smaller than the reference value for each reference time, the blink control unit MCC transmits spray control signals to the first and second spray driving units EZ1 and EZ2. Accordingly, the first and second spray driving units EZ1 and EZ2 spray at least one of air, water, and the tear solution to the user's left and right eyes, respectively, during a period in which the spray control signals are input from the blink control unit MCC (SS4).

When the number of inputs of the blink sensing signals for each reference time is detected to be smaller than the reference value for each reference time, the blink control unit MCC transmits an eyeball protection operation execution notification message or a fatigue increase message to the display control unit PCM. The eyeball protection operation execution notification message may be displayed on the display panel DP under the control of the display control unit PCM (SS5).

The first and second temperature detection units ER1 and ER2 sense temperatures of the user's left and right eyes, respectively, in real time from a point in time initialized by the blink control unit MCC and output temperature sensing signals of the user's left and right eyes, respectively. The temperature sensing signals of the user's left and right eyes output from the first and second temperature detection units ER1 and ER2 are transmitted to the blink control unit MCC. The blink control unit MCC detects temperature values of the user's left and right eyes from the temperature sensing signals of the user's left and right eyes received through the first and second temperature detection units ER1 and ER2 (SS6).

The blink control unit MCC compares the temperature values of the user's left and right eyes with a threshold temperature value (SS7).

When the temperature values of the user's left and right eyes become greater than the threshold temperature value, the blink control unit MCC transmits spray control signals to the first and second spray driving units EZ1 and EZ2 (SS8). Accordingly, the first and second spray driving units EZ1 and EZ2 spray at least one of air, water, and the tear solution to the user's left and right eyes, respectively, during a period in which the spray control signals are input from the blink control unit MCC.

When the temperature values of the user's left and right eyes become greater than the threshold temperature value, the blink control unit MCC transmits an eyeball protection operation execution notification message to the display control unit PCM. The eyeball protection operation execution notification message may be displayed on the display panel DP under the control of the display control unit PCM (SS5).

The use time detection unit CMT detects a point in time at which the image data or the control signals from the display control unit PCM are stopped without being transmitted to the display panel DP, that is, a point in time at which an image display operation of the display panel DP ends. When the image display operation of the display panel DP ends, the use time detection unit CMT supplies an image display operation end signal of the display panel DP to the blink control unit MCC. The blink control unit MCC may end a control operation of the first and second blink sensing units EC1 and EC2 and the first and second temperature detection units ER1 and ER2 and may initialize operations of the first and second blink sensing units EC1 and EC2 and the first and second temperature detection units ER1 and ER2 again (SS6).

The use time detection unit CMT may monitor an image display period of the display panel DP from an initialization point in time initialized by the blink control unit MCC and may accumulate and output information on a gaze period during which the user gazes at the displayed image. The use time detection unit CMT counts a period during which the image data or the control signals from the display control unit PCM are transmitted to the display panel DP with the count circuit and accumulates information on a period during which an image is displayed on the display panel DP. In addition, the use time detection unit CMT transmits the accumulated information on the gaze period to the blink control unit MCC in units of a period.

The blink control unit MCC receives the accumulated information on the gaze period during which the user gazes at the displayed image in real time from the use time detection unit CMT. In addition, the blink control unit MCC compares the information on the gaze period of the user with use time reference information for each step.

When the information on the gaze period of the user becomes the same as the use time reference information for each step, the blink control unit MCC may transmit spray control signals to the first and second spray driving unit EZ1 and EZ2. In addition, the blink control unit MCC may transmit an eyeball protection operation execution notification message and a fatigue increase message to the display control unit PCM.

As described above, the eyeball protection module ECM included in the wearable device 1 may induce user's blink to protect the user's eyes by checking (or determining) a user's blink period and eyeball temperature and spraying air, water, the tear solution, or the like to the user's eyes. In addition, the eyeball protection module ECM may allow a fatigue increase message according to a change in the user's blink period and eyeball temperature to be displayed as an image.

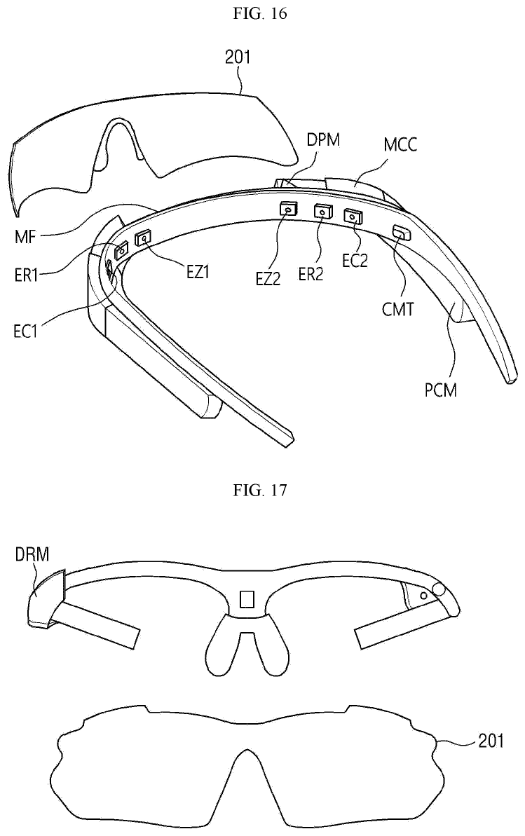

FIG. 15 is an exploded perspective view illustrating a wearable device including an image display module according to another embodiment of the present disclosure, FIG. 16 is an exploded perspective view, in a rear direction, of an augmented reality providing device illustrated in FIG. 15, and FIG. 17 is an exploded perspective view, in a front direction, of the augmented reality providing device illustrated in FIGS. 15 and 16.

Referring to FIGS. 15 to 17, a wearable device 1 according to another embodiment may be a see-through type augmented reality (AR) providing device formed in the shape of glasses.

The wearable device 1 providing augmented reality includes a main frame MF supporting one or more transparent lens 201 to be mounted on a user's body and an image display module DPM mounted on the main frame MF for displaying an image through the transparent lens 201. In addition, the wearable device 1 includes an eyeball protection module ECM operating to protect the user's eyeballs in a rear direction or a wearing direction of the main frame MF, in other words, in a direction in which the main frame MF is worn on the user's face or eye parts.

The main frame MF may be formed in the shape of glasses including a glasses frame supporting an edge of the transparent lens 201 and glasses temples. The shape of the main frame MF is not limited to the shapes of glasses and may have, in other embodiments, a goggle shape or a head mount shape including the transparent lens 201. A display control unit PCM for controlling overall operations of the image display module DPM may be disposed inside the main frame MF or on one side surface of the main frame MF.

The transparent lens 201 may be formed integrally in left and right directions or may include first and second transparent lenses separated from each other in the left and right directions. In either embodiment, the transparent lens 201 may be transparent or translucent and formed of glass or plastic. Accordingly, the user may see a real image through the transparent lens 201. In either embodiment, the transparent lens 201 may have a refractive power in consideration of a user's eyesight.

The transparent lens 201 may further include one or more reflective members reflecting an augmented reality (AR) content image provided from the image display module DPM toward the transparent lens 201 or the user's eyes and optical members adjusting a focus and a size. One or more reflective members may be embedded in the transparent lens 201 integrally with the transparent lens 201 and may be formed as a plurality of refractive lenses or a plurality of prisms having a curvature (e.g., a predetermined curvature).

The image display module DPM may include a micro LED display device (micro-LED), a nano LED display device (nano-LED), an organic light emitting display device (OLED), an inorganic light emitting display device (inorganic EL), a quantum dot light emitting display device (QED), a cathode ray display (CRT), a liquid crystal display (LCD), and the like. Hereinafter, an embodiment in which a micro LED display device is included in the image display module DPM will be described as an example, and unless a special distinction is required, the micro LED display device will be simply referred to as a display device. However, an embodiment is not limited to the micro LED display device, and other display devices listed above or known in the technical field may also be applied within the scope of the technical idea.

As described above, the eyeball protection module ECM includes the first and second blink sensing units EC1 and EC2, the first and second temperature detection units ER1 and ER2, the use time detection unit CMT, the first and second spray driving units EZ1 and EZ2, and the blink control unit MCC.

The first and second blink detection units EC1 and EC2, the first and second temperature detection units ER1 and ER2, and the first and second spray driving units EZ1 and EZ2 may be disposed, respectively, at positions facing user's eyeball positions, that is, on an inner mounting surface of the main frame MF. For example, the first and second blink sensing units EC1 and EC2 may be formed on an inner surface of the main frame MF facing the user's eyeball positions and may generate and output blink sensing signals according to blink motions of the user's left and right eyes. All of the first and second temperature detection units ER1 and ER2 and the first and second spray driving units EZ1 and EZ2 may be mounted on the main frame MF or the transparent lens 201 at positions adjacent to the first and second blink sensing units EC1 and EC2.

The blink control unit MCC may be formed in a one-chip type with the display control unit PCM; that is, the blink control unit MCC may be integrated with the display control unit PCM and may be disposed inside the main frame MF or on one side surface of the main frame MF. For example, the display control unit PCM and the blink control unit MCC may be assembled to at least one side of the main frame MF together with at least one image display module DPM or may be formed integrally with the main frame MF. The display control unit PCM supplies augmented reality content data to the image display module DPM so that the image display module DPM displays an augmented reality content, for example, an augmented reality content image.

The blink control unit MCC is configured to control spray driving of the first and second spray driving units EZ1 and EZ2 based on at least one signal or information of the blink sensing signals of the user's left and right eyes, the temperature sensing signals for the user's left and right eyes, and the information on the gaze period during which the user gazes at the displayed image.

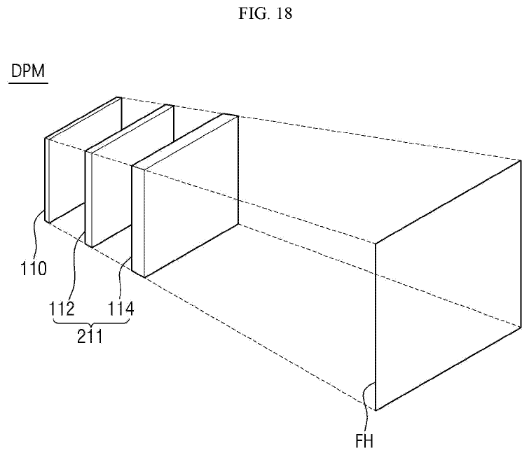

FIG. 18 is a schematic exploded perspective view illustrating the image display module illustrated in FIGS. 15 to 17.

Referring to FIG. 18, the image display module DPM for displaying the augmented reality content image may be assembled to one side or both sides of the main frame MF or may be formed integrally with the main frame MF.

The image display module DPM allows the augmented reality content image to be viewed by the user in a form in which the augmented reality content image is superimposed on a real image visible to the user through the transparent lens 201 by allowing the augmented reality content image to be displayed on the transparent lens 201. To this end, the image display module DPM includes an image display device 110 for displaying the augmented reality content image and an image transmission member transmitting the augmented reality content image to the transparent lens 201. Here, the image transmission member 211 may include at least one of suitable optical members, such as an optical waveguide (e.g., a prism), a diffusion lens 112, and a focusing lens 114. Accordingly, the augmented reality content image displayed through each image display device 110 may be provided to the transparent lens 201 and the user's eyes through the optical waveguide, the diffusion lens 112, the focusing lens 114, and the like.

The image display device 110 included in the image display module DPM may include a micro LED display device (micro-LED), a nano LED display device (nano-LED), an organic light emitting display device (OLED), an inorganic light emitting display device (inorganic EL), a quantum dot light emitting display device (QED), and the like. Hereinafter, an embodiment in which the image display device 110 includes a micro LED display device will be described. However, the present disclosure is not limited to the micro LED display device, and other display devices listed above or known in the technical field may also be applied within the scope of the technical idea.

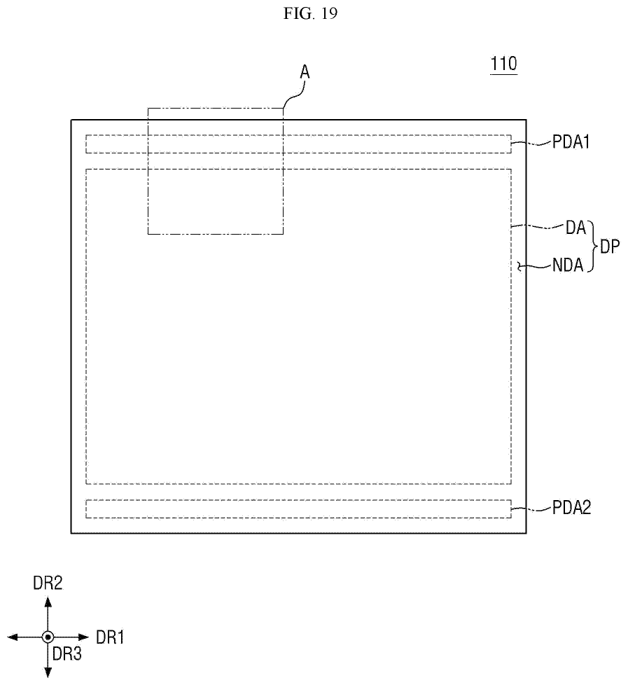

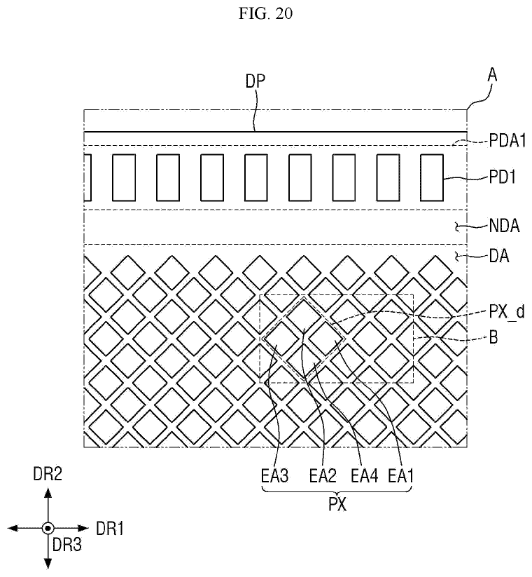

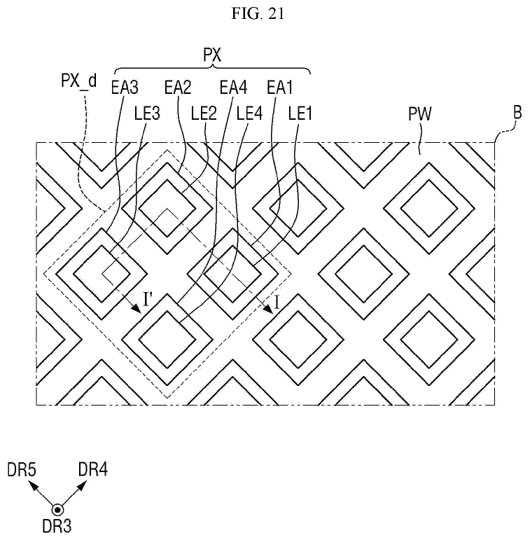

FIG. 19 is a layout diagram illustrating an image display device illustrated in FIG. 18, FIG. 20 is a layout diagram illustrating the area A of FIG. 19, and FIG. 21 is a layout diagram illustrating pixels illustrated in the area B of FIG. 20.

It has been illustrated by way of example that the image display device 110 according to an embodiment illustrated in FIGS. 19 to 21 has a light emitting diode on silicon (LEDoS) structure in which light emitting diode elements are disposed on a semiconductor circuit board formed through a semiconductor process. However, the present disclosure is not limited thereto. In addition, it has been primarily described that the image display device 110 according to an embodiment of the present disclosure is a micro light emitting diode display module (or a nano light emitting diode display module) including micro light emitting diodes (or nano light emitting diodes) as light emitting elements, but the present disclosure is not limited thereto.

In FIGS. 19 to 21, a first direction DR1 refers to a transverse direction of the image display device 110, a second direction DR2 refers to a longitudinal direction of the image display device 110, and a third direction DR3 refers to a thickness direction of a display panel DP or a thickness direction of a semiconductor circuit substrate 215. In addition, a fourth direction DR4 refers to a diagonal direction of the display panel DP, and the fifth direction DR5 refers to a diagonal direction crossing the fourth direction DR4. Here, the terms “left”, “right”, “upper”, and “lower” refer to directions when the display device DP is viewed in a plan view. For example, “right side” refers to one side in the first direction DR1, “left side” refers to the other side in the first direction DR1, “upper side” refers to one side in the second direction DR2, and “lower side” refers to the other side in the second direction DR2. In addition, “upper portion” refers to one side in the third direction DR3, and “lower portion” refers to the other side in the third direction DR3.

Referring to FIGS. 19 to 21, the image display device 110 includes a display panel DP having a display area DA and a non-display area NDA.

The display panel DP of the image display device 110 may have a rectangular shape, in a plan view, having long sides in the first direction DR1 and short sides in the second direction DR2. However, the shape of the display panel DP in a plan view is not limited thereto, and the display panel DP may have a polygonal shape other than the rectangular shape, a circular shape, an elliptical shape, or an irregular shape in a plan view.

The display area DA may be an area in which an image is displayed, and the non-display area NDA may be an area in which an image is not displayed. A shape of the display area DA in a plan view may follow (or may correspond to) the shape of the display panel DP in a plan view. In FIG. 19, an embodiment in which the shape of the display area DA in a plan view is a rectangular shape is illustrated. The display area DA may be disposed in a central area of the display panel DP. The non-display area NDA may be disposed around (e.g., around a periphery of) the display area DA. The non-display area NDA may be disposed to surround (e.g., to surround in a plan view) the display area DA.

A first pad part PDA1 may be disposed in the non-display area NDA. The first pad part PDA1 may be disposed on the upper side of the display panel DP. The first pad part PDA1 may include first pads PD1 connected to an external circuit board. A second pad part PDA2 may also be disposed in the non-display area NDA. The second pad part PDA2 may be disposed on the lower side of the semiconductor circuit substrate. The second pad part PDA2 may include second pads to be connected to the external circuit board. In some embodiments, the second pad part PDA2 may be omitted.

The display area DA of the display panel DP may include a plurality of pixels PX. Each pixel PX may be defined as a minimum light emitting unit for displaying white light in each defined pixel area PX_d.

The pixels PX, disposed as minimum units for displaying white light in the respective pixel areas PX_d, may include a plurality of emission areas EA1, EA2, EA3, and EA4. In an embodiment of the present disclosure, the respective pixels PX include four emission areas EA1, EA2, EA3, and EA4 disposed in a Pentile® (a registered trademark of Samsung Display Co., Ltd.) (or diamond) matrix structure, but the present disclosure is not limited thereto. For example, each of the plurality of pixels PX may include only three emission areas EA1, EA2, and EA3.

The plurality of emission areas EA1, EA2, EA3, and EA4 for each pixel area PX_d may be partitioned by a partition wall PW. The partition wall PW may be disposed to surround (e.g., to surround in a plan view) each of first to fourth light emitting elements LE1 to LE4 disposed in the emission areas EA1, EA2, EA3, and EA4. The partition wall PW may be disposed to be spaced apart from each of the first to fourth light emitting elements LE1 to LE4. The partition wall PW may have a mesh shape, a net shape, or a lattice shape in a plan view.

In FIGS. 20 and 21, each of the plurality of emission areas EA1, EA2, EA3, and EA4 defined by the partition wall PW is illustrated as having a rhombic shape, in a plan view, forming the Pentile® matrix structure, but the present disclosure is not limited thereto. For example, each of the plurality of emission areas EA1, EA2, EA3, and EA4 defined by the partition wall PW may have a polygonal shape, such as a quadrangular shape or a triangular shape other than the rhombic shape, a circular circuit, an elliptical shape, or an irregular shape.

Referring to FIG. 21, a first emission area EA1 from among the plurality of emission areas EA1, EA2, EA3 and EA4 may include a first light emitting element LE1 for emitting first light, a second emission area EA2 from among the plurality of emission areas EA1, EA2, EA3 and EA4 may include a second light emitting element LE2 for emitting second light, a third emission area EA3 from among the plurality of emission areas EA1, EA2, EA3 and EA4 may include a third light emitting element LE3 for emitting third light, and a fourth emission area EA4 from among the plurality of emission areas EA1, EA2, EA3 and EA4 may include a fourth light emitting element LE4 for emitting fourth light. The first light may be light of a wavelength band providing any one of red, green, and blue color light. In addition, the second light may be light of a wavelength band implementing any one color different from that of the first light from among the red, the green, and the blue. The third light may be light of a wavelength band implementing any one color different from those of the first light and the second light from among the red, the green, and the blue. In addition, the fourth light may be light of the same wavelength band as any one of the first light to the third light.

Each of the first to fourth light emitting elements LE1 to LE4 included in the first to fourth emission areas EA1 to EA4 disposed in the Pentile® matrix structure has been illustrated as having a rhombic shape in a plan view, but the present disclosure is not limited thereto. For example, each of the first to fourth light emitting elements LE1 to LE4 may be formed in a polygonal shape, such as a triangular shape or a quadrangular shape other than the rhombus shape, a circular shape, an elliptical shape, or an irregular shape.

Each of the first emission areas EA1 refers to an area for emitting the first light. Each of the first emission areas EA1 outputs the first light emitted from the first light emitting element LE1. As described above, the first light may be the light of the wavelength band implementing any one of the red, the green, and the blue. As an example, the first light may be light of a red wavelength band. The red wavelength band may be in a range of approximately 600 nm to approximately 750 nm, but the present disclosure is not limited thereto.

Each of the second emission areas EA2 refers to an area for emitting the second light. Each of the second emission areas EA2 outputs the second light emitted from the second light emitting element LE2. The second light may be the light of the wavelength band implementing any one color different from that of the first light from among the red, the blue, and the green. As an example, the second light may be light of a blue wavelength band. The blue wavelength band may be in a range of approximately 370 nm to approximately 460 nm, but the present disclosure is not limited thereto.

Each of the third emission areas EA3 refers to an area for emitting the third light. Each of the third emission areas EA3 outputs the third light emitted from the third light emitting element LE3. The third light may be the light of the wavelength band implementing any one color different from those of the first light and the second light from among the red, the blue, and the green. As an example, the third light may be light of a green wavelength band. The green wavelength band may be in a range of approximately 480 nm to approximately 560 nm, but the present disclosure is not limited thereto.

Each of the fourth emission areas EA4 refers to an area for emitting the fourth light. Each of the fourth emission areas EA4 outputs the fourth light emitted from the fourth light emitting element LE4. Here, the fourth light may be light implementing the same color as any one of the first light to the third light. As an example, the fourth light may be light of a blue wavelength band that is the same as the second light or may be light of a green wavelength band that is the same as the third light. However, the present disclosure is not limited thereto.

The second emission areas EA2 of the respective pixels PX may be alternately disposed with the fourth emission areas EA4 of other adjacent pixels PX along the first direction DR1, which is a transverse (or row) direction. In addition, the first emission areas EA1 and the third emission areas EA3 of the respective pixels PX may be alternately disposed along the first direction DR1, which is the transverse (or row) direction. The fourth emission areas EA4 of the respective pixels PX may be alternately disposed with the second emission areas EA2 of other adjacent pixels PX along the first direction DR1, which is the transverse (or row) direction.

The first emission areas EA1 and the fourth emission areas EA4 are alternately disposed in the fourth direction DR4, which is a first diagonal direction, and the second emission areas EA2 and the third emission areas EA3 are also alternately disposed in the fourth direction DR4, which is the first diagonal direction. Accordingly, the second emission areas EA2 and the first emission areas EA1 are alternately disposed in the fifth direction DR5, which is a second diagonal direction crossing the first diagonal direction, and the third emission areas EA3 and the fourth emission areas EA4 are also alternately disposed in the fifth direction DR5, which is the second diagonal direction, such that the respective pixels PX may also be disposed and arranged in the Pentile® matrix structure as a whole.

Sizes or planar areas of the first to fourth emission areas EA1 to EA4 of each pixel PX may be the same as or different from each other. Likewise, sizes or planar areas of the first to fourth light emitting elements LE1 to LE4, formed in the first to fourth emission areas EA1 to EA4, respectively, may be the same as or different from each other.

An area of the first emission area EA1, an area of the second emission area EA2, an area of the third emission area EA3, and an area of the fourth emission area EA4 may be substantially the same as each other, but the present disclosure is not limited thereto. For example, areas of the first and second emission areas EA1 and EA2 may be different from each other, areas of the second and third emission areas EA2 and EA3 may also be different from each other, and areas of the third and fourth emission areas EA3 and EA4 may also be different from each other. Areas of at least two pairs of the first to fourth emission areas EA1 to EA4 may be the same as each other.

A distance between the first and second emission areas EA1 and EA2 neighboring to each other in a horizontal direction or the diagonal direction, a distance between the second and third emission areas EA2 and EA3 neighboring to each other in the horizontal direction or the diagonal direction, a distance between the third and fourth emission areas EA3 and EA4 neighboring to each other in the horizontal direction or the diagonal direction, and a distance between the first and fourth emission areas EA1 and EA4 neighboring to each other in the horizontal direction or the diagonal direction may be the same as each other but may be different from each other according to different areas. The present disclosure is not limited thereto.

The present disclosure is not limited to an example in which the first emission area EA1 emits the first light, the second emission area EA2 emits the second light, the third emission area EA3 emits the third light, and the fourth emission area EA4 emits the same light as any one of the first light to the third light. At least one of the first to fourth emission areas EA1 to EA4 may also emit fifth light. Here, the fifth light may be light of a yellow wavelength band. That is, a main peak wavelength of the fifth light may be in a range of approximately 550 nm to approximately 600 nm, but the present disclosure is not limited thereto.

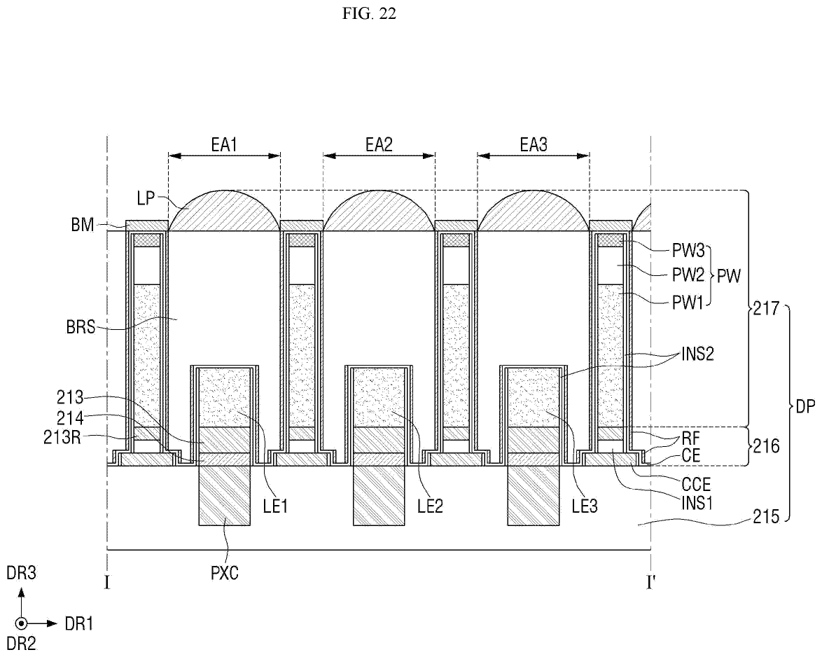

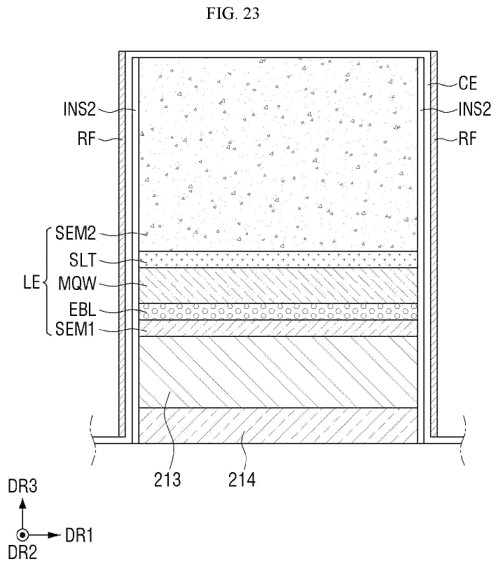

FIG. 22 is a cross-sectional view illustrating the image display device taken along the line I-I′ of FIG. 21, and FIG. 23 is an enlarged cross-sectional view of a light emitting element shown in FIG. 22.

Referring to FIGS. 22 and 23, the display panel DP may include the semiconductor circuit substrate 215, a conductive connection layer 216, and a light emitting element layer 217.

The semiconductor circuit substrate 215 may include a plurality of pixel circuit parts PXC and pixel electrodes 214. The conductive connection layer 216 may include connection electrodes 213, first pads PD1, a common connection electrode CCE, a first insulating layer INS1, and a conductive pattern 213R.

The semiconductor circuit substrate 215 may be a silicon wafer substrate formed by using a semiconductor process. The plurality of pixel circuit parts PXC of the semiconductor circuit substrate 215 may be formed by using a semiconductor process.

The plurality of pixel circuit parts PXC may be disposed in the display area DA (see, e.g., FIG. 19). Each of the plurality of pixel circuit parts PXC may be connected to the pixel electrode 214 corresponding thereto. For example, the plurality of pixel circuit parts PXC and a plurality of pixel electrodes 214 may be connected to each other in one-to-one correspondence. Each of the plurality of pixel circuit parts PXC may overlap one of the corresponding light emitting elements LE1 to LE4 in the third direction DR3. Various other modified circuit structures, such as a 3T1C structure, a 2T1C structure, a 7T1C structure, and a 6T1C structure, may be applied to each of the pixel circuit parts PXC.

Each of the pixel electrodes 214 may be disposed on the pixel circuit part PXC corresponding thereto. Each of the pixel electrodes 214 may be an exposed electrode exposed from the pixel circuit part PXC. For example, each of the pixel electrodes 214 may protrude from an upper surface of the pixel circuit part PXC. Each of the pixel electrodes 214 may be formed integrally with the pixel circuit part PXC. Each of the pixel electrodes 214 may receive a pixel voltage (e.g., an anode voltage) supplied from the pixel circuit part PXC. The pixel electrodes 214 may be made of aluminum (Al).

Each of the connection electrodes 213 may be disposed on the pixel electrode 214 corresponding thereto. Each of the connection electrodes 213 may be disposed on the pixel electrode 214. The connection electrodes 213 may include a metal material for adhering the pixel electrodes 214 to the respective light emitting elements LE1 to LE4.

The common connection electrode CCE may be disposed to be spaced apart from the pixel electrodes 214 and the connection electrodes 213. The common connection electrode CCE may be disposed to surround the pixel electrodes 214 and the connection electrodes 213. The common connection electrode CCE may be connected to any one of the first pads PD1 of the first pad part PDA1 of the non-display area NDA to receive a common voltage. The common connection electrode CCE may include the same material as the connection electrodes 213.

The first insulating layer INS1 may be disposed on the common connection electrode CCE. A width of the first insulating layer INS1 in the first direction DR1 or the second direction DR2 may be smaller than a width of the common connection electrode CCE. Accordingly, a portion of an upper surface of the common connection electrode CCE is not covered by the first insulating layer INS1 and may be exposed therethrough. A portion of the upper surface of the common connection electrode CCE that is not covered by the first insulating layer INS1 (e.g., is exposed therethrough) may be in contact with a common electrode CE. Therefore, the common electrode CE may be connected to the common connection electrode CCE.

The conductive pattern 213R may be disposed on the first insulating layer INS1. The conductive pattern 213R may be disposed between the first insulating layer INS1 and the partition wall PW. A width of the conductive pattern 213R may be substantially the same as the width of the first insulating layer INS1 or a width of the partition wall PW. The conductive pattern 213R corresponds to a residue formed by the same process as a process of forming the connection electrodes 213 and the common connection electrode CCE.

The light emitting element layer 217 may include the respective light emitting elements LE1, LE2, LE3, and LE4, the partition wall PW, a second insulating layer INS2, the common electrode CE, a reflective layer RF, a light blocking member BM, and optical patterns LP.

The light emitting element layer 217 may include the first to fourth emission areas EA1 to EA4 partitioned by the partition wall PW. At least one of each light emitting element LE and optical pattern LP may be disposed in each of the first to fourth emission areas EA1 to EA4.

The light emitting elements LE1, LE2, and LE3 shown in FIG. 22 may be disposed on the connection electrodes 213 in the emission areas EA1 to EA3, respectively. A length (or a height) of each of the light emitting elements LE1, LE2, and LE3 in the third direction DR3 may be greater than a length thereof in the horizontal direction. The length in the horizontal direction refers to a length in the first direction DR1 or a length in the second direction DR2. For example, a length of the first light emitting element LE1 in the third direction DR3 may be in a range of approximately 1 μm to approximately 5 μm.

Referring to FIG. 23, each of the light emitting elements LE1, LE2, LE3, and LE4 includes a first semiconductor layer SEM1, an electron blocking layer EBL, an active layer MQW, a superlattice layer SLT, and a second semiconductor layer SEM2. The first semiconductor layer SEM1, the electron blocking layer EBL, the active layer MQW, the superlattice layer SLT, and the second semiconductor layer SEM2 may be sequentially stacked in the third direction DR3.

The first semiconductor layer SEM1 may be disposed on the connection electrode 213. The first semiconductor layer SEM1 may be a semiconductor layer doped with a first conductivity-type dopant, such as Mg, Zn, Ca, Se, or Ba. For example, the first semiconductor layer SEM1 may be made of p-GaN doped with p-type Mg. A thickness of the first semiconductor layer SEM1 may be in a range of approximately 30 nm to approximately 200 nm.

The electron blocking layer EBL may be disposed on the first semiconductor layer SEM1. The electron blocking layer EBL may be a layer for suppressing or preventing too many electrons from flowing to the active layer MQW. For example, the electron blocking layer EBL may be made of p-AlGaN doped with p-type Mg. A thickness of the electron blocking layer EBL may be in a range of approximately 10 nm to approximately 50 nm. In some embodiments, the electron blocking layer EBL may be omitted.

The active layer MQW may be divided into first to third active layers. Each of the first to third active layers may include a material having a single or multiple quantum well structure. When each of the first to third active layers includes the material having the multiple quantum well structure, each of the first to third active layers may have a structure in which a plurality of well layers and barrier layers are alternately stacked. In such an embodiment, the first active layer may include InGaN or GaAs, and the second active layer and the third active layer may include InGaN, but the present disclosure is not limited thereto. The first active layer may emit light by a combination of electron-hole pairs according to an electrical signal. The first active layer may emit first light having a main peak wavelength in a range of approximately 600 nm to approximately 750 nm, that is, light of a red wavelength band. The second active layer may emit light by a combination of electron-hole pairs according to an electrical signal. The second active layer may emit third light having a main peak wavelength in a range of approximately 480 nm to approximately 560 nm, that is, light of a green wavelength band. The third active layer may emit light by a combination of electron-hole pairs according to an electrical signal. The third active layer may emit second light having a main peak wavelength in a range of approximately 370 nm to approximately 460 nm, that is, light of a blue wavelength band.

A color of the light emitted from each of the first to third active layers may be changed according to a content of indium in each of the first to third active layers. For example, as the content of indium decreases, a wavelength band of the light emitted from each of the first to third active layers may move to a red wavelength band, and as the content of indium increases, a wavelength band of the light emitted from each of the first to third active layers may move to a blue wavelength band. The content of indium (In) in the first active layer may be higher than that of indium (In) in the second active layer, and the content of indium (In) in the second active layer may be higher than that of indium (In) in the third active layer. For example, the content of indium (In) in the third active layer may be about 15%, the content of indium (In) in the second active layer may be about 25%, and the content of indium (In) in the first active layer may be about 35% or higher.

Because the color of emitted light may be changed according to the content of indium in each of the first to third active layers, the light emitting element layers 217 of the respective light emitting elements LE1, LE2, and LE3 may emit light, such as the first light, the second light, and the third light that are the same as or different from each other, according to the content of indium. For example, when the content of indium (In) in the first to third active layers of the first light emitting element LE1 are within about 15%, the first light emitting element LE1 may emit the first light of the red wavelength band having the main peak wavelength in a range of approximately 600 nm to approximately 750 nm. In addition, when the content of indium (In) in the first to third active layers of the second light emitting element LE2 are about 25%, the second light emitting element LE2 may emit the second light of the green wavelength band having the main peak wavelength in a range of approximately 480 nm to approximately 560 nm. In addition, when the content of indium (In) in the first to third active layers of the third light emitting element LE3 are about 35% or higher, the third light emitting element LE3 may emit the third light of the blue wavelength band having the main peak wavelength in a range of approximately 370 nm to approximately 460 nm. By adjusting and setting the content of indium (In) in the first to third active layers of the fourth light emitting element LE4, the fourth light emitting element LE4 may also emit the first to third lights or may emit another fourth light.

The superlattice layer SLT may be disposed on the active layer MQW. The superlattice layer SLT may be a layer for alleviating stress between the second semiconductor layer SEM2 and the active layer MQW. For example, the superlattice layer SLT may be made of InGaN or GaN. A thickness of the superlattice layer SLT may be in a range of approximately 50 nm to approximately 200 nm. In some embodiments, the superlattice layer SLT may be omitted.

The second semiconductor layer SEM2 may be disposed on the superlattice layer SLT. The second semiconductor layer SEM2 may be doped with a second conductivity-type dopant, such as Si, Ge, or Sn. For example, the second semiconductor layer SEM2 may be made of n-GaN doped with n-type Si. A thickness of the second semiconductor layer SEM2 may be in a range of approximately 2 μm to approximately 4 μm.

The partition wall PW may be disposed to be spaced apart from each of the light emitting elements LE1 to LE4 disposed in each of the first to fourth emission areas EA1 to EA4. The partition wall PW may be disposed to surround the light emitting elements LE1 to LE4 each disposed in the first to fourth emission areas EA1 to EA4.

The partition wall PW may be disposed on the common connection electrode CCE. A width of the partition wall PW in the first direction DR1 and the second direction DR2 may be smaller than the width of the common connection electrode CCE. The partition wall PW may be disposed to be spaced apart from the light emitting elements LE.

The partition wall PW may include a first partition wall PW1, a second partition wall PW2, and a third partition wall PW3. The first partition wall PW1 may be disposed on the first insulating layer INS1. The first partition wall PW1 may be formed by the same process as a process of the light emitting element LE, and thus, at least a portion of the first partition wall PW1 may include the same material as the light emitting element LE.

The second insulating layer INS2 may be disposed on side surfaces of the common connection electrode CCE, side surfaces of the partition wall PW, side surfaces of each of the pixel electrodes 214, side surfaces of each of the connection electrodes 213, and side surfaces of each of the light emitting elements LE1 to LE4. The second insulating layer INS2 may be formed as an inorganic layer, such as a silicon oxide (e.g., SiO₂) layer. A thickness of the second insulating layer INS2 may be about 0.1 μm.

The common electrode CE may be disposed on an upper surface and the side surfaces of each of the light emitting elements LE1 to LE4 and an upper surface and the side surfaces of the partition wall PW. That is, the common electrode CE may be disposed to cover the upper surface and the side surfaces of each of the light emitting elements LE1 to LE4 and the upper surface and the side surfaces of the partition wall PW.

The common electrode CE may be in contact with the second insulating layer INS2 disposed on the side surfaces of the common connection electrode CCE, the side surfaces of the partition wall PW, the side surfaces of each of the pixel electrodes 214, the side surfaces of each of the connection electrodes 213, and the side surfaces of each of the light emitting elements LE1 to LE4. In addition, the common electrode CE may be in contact with the upper surface of the common connection electrode CCE, the upper surface of each of the light emitting elements LE1 to LE4, and the upper surface of the partition wall PW.

The common electrode CE may be in contact with the upper surface of the common connection electrode CCE and the upper surfaces of the light emitting elements LE1 to LE4 that are not covered by the second insulating layer INS2 and are exposed. Therefore, the common voltage supplied to the common connection electrode CCE may be applied to the light emitting elements LE1 to LE4. That is, one end of the light emitting elements LE1 to LE4 may receive the pixel voltage (or the anode voltage) of the pixel electrodes 214 through the connection electrodes 213, and the other end of the light emitting elements LE1 to LE4 may receive the common voltage through the common electrode CE. The light emitting element LE may emit light with a luminance according to a voltage difference between the pixel voltage and the common voltage.

The reflective layer RF may be disposed on the side surfaces of the common connection electrode CCE, the side surfaces of the partition wall PW, the side surfaces of each of the pixel electrodes 214, the side surfaces of each of the connection electrodes 213, and the side surfaces of each of the light emitting elements LE1 to LE4. The reflective layer RF reflects light traveling toward left and right side surfaces rather than an upwardly direction from among the light emitted from the light emitting elements LE1 to LE4. The reflective layer RF may include a metal material having high reflectivity, such as aluminum (Al). A thickness of the reflective layer RF may be about 0.1 μm.

A base resin BRS may be disposed on a passivation layer PTF in each of the light emitting elements LE1 to LE4. The base resin BRS may include a light-transmitting organic material. The base resin BRS may further include scatterers for scattering the light of the light emitting elements LE1 to LE4 in random directions. The scatterers may include metal oxide particles or organic particles.

The light blocking member BM may be disposed on the partition wall PW. The light blocking member BM may include a light blocking material. The light blocking member BM may be disposed between the respective emission areas EA1, EA2, EA3 and EA4 adjacent to each other to prevent color mixing of light of different wavelength bands emitted from the light emitting elements LE1 to LE4 of the respective emission areas EA1, EA2, EA3, and EA4. In addition, the light blocking member BM may reduce external light reflection by absorbing at least some of external light incident from the outside onto the light emitting element layer 217. The light blocking member BM may be positioned on the partition wall PW and may be disposed to further extend to the respective emission areas EA1, EA2, EA3, and EA4. For example, a width of the light blocking member BM may be greater than the width of the partition wall PW.

Each of the optical patterns LP may be selectively disposed on each of the emission areas EA1, EA2, EA3, and EA4. Each of the optical patterns LP may be directly disposed on the base resin BRS of each of the emission areas EA1, EA2, EA3, and EA4. The optical patterns LP may have a shape in which they protrude in an upwardly direction (e.g., a direction from the light emitting elements LE1 to LE4 toward the respective optical patterns LP). For example, a cross section of each of the optical patterns LP may have an upwardly convex lens shape. Each of the optical patterns LP may be disposed on the base resin BRS and the light blocking member BM disposed therebelow. A width of each optical pattern LP may be equal to, greater than, or smaller than a width of each of the emission areas EA1, EA2, EA3, and EA4. The respective optical patterns LP may collect the first light to the third light or the fourth light transmitted through the base resin BRS in the respective emission areas EA1, EA2, EA3, and EA4.

As described above, the wearable device 1 may be a see-through type head mounted display that provides augmented reality (AR) or may be a see-closed type head mounted display that provides virtual reality (VR). The eyeball protection module ECM may be disposed in an inner direction of such a wearable device 1, that is, in a direction in which the wearable device 1 is mounted on the user's face or eye areas to protect the user's eyeballs. The eyeball protection module ECM may spray at least one of air, water, and a tear solution to the user's eyeballs based on at least one signal or information of the blink sensing signals of the user's left and right eyes, the temperature sensing signals for the user's left and right eyes, and the information on the gaze period during which the user gazes at the displayed image.

In concluding the detailed description, those skilled in the art will appreciate that many variations and modifications can be made to the embodiments described herein without substantially departing from the present disclosure. Therefore, the described embodiments of the present disclosure are to be used in a generic and descriptive sense and not for purposes of limitation.

What is claimed is:

1. A wearable device comprising:
a main frame configured to be mounted on a user's body;
an image display module on the main frame and configured to display an image;
a lens frame on an image display surface of the image display module, the lens frame being configured to refract image display light emitted from the image display module;
a multi-channel lens forming an emission path of the image display light refracted by the lens frame for each of a plurality of channels; and
an eyeball protection module on the main frame and configured to spray air, moisture, or a tear solution to the user's left and right eyes.

2. The wearable device of claim 1, wherein the eyeball protection module comprises:
first and second blink sensing units configured to sense blink motions of the user's left and right eyes in real time and to generate and outputting blink sensing signals of the user's left and right eyes;
first and second temperature detection units configured to sense temperatures of the user's left and right eyes in real time and to output temperature sensing signals of the user's left and right eyes;
first and second spray driving units configured to spray the air, water, or tear solution to the user's left and right eyes to induce user's blink motions; and
a blink control unit configured to control spray driving of the first and second spray driving units based on at least one of the blink sensing signals of the user's left and right eyes and the temperature sensing signals for the user's left and right eyes.

3. The wearable device of claim 2, wherein the first and second blink sensing unit comprise:
an image sensor configured to capture images of the user's left and right eyes in real time;
an image analyzer configured to compare the captured eyeball images with each other in units of at least one frame and to detect eyelid images as a comparison result; and
a blink sensing signal output unit configured to generate blink sensing signals according to the detection of the eyelid images and to supply the blink sensing signals to the blink control unit.

4. The wearable device of claim 2, wherein the first and second temperature detection units comprise:
a contactless thermometer configured to sense the temperatures of the user's left and right eyes in real time; and
an analog-to-digital converter configured to transmit the temperature sensing signals corresponding to the sensed temperatures of the user's left and right eyes to the blink control unit.

5. The wearable device of claim 2, wherein the first and second spray driving units comprise:
a storage capsule for storing the air, water, or tear solution; and
a spray pump configured to spray the air, water, or tear solution to the user's left and right eyes during a period in which a spray control signal is input from the blink control unit.

6. The wearable device of claim 2, wherein the blink control unit is configured to:
detect a number of inputs of the blink sensing signals of the user's left and right eyes in units of a reference time;
compare the number of inputs of the blink sensing signals for each reference time unit with a reference value for each reference time; and
when the number of inputs of the blink sensing signals for each reference time unit is determined to be smaller than the reference value for each reference time, transmit spray control signals to the first and second spray driving units.

7. The wearable device of claim 6, wherein, when the number of inputs of the blink sensing signals for each reference time unit is determined to be smaller than the reference value for each reference time unit, the blink control unit is configured to transmit an eyeball protection operation execution notification message to a display control unit of the image display module to allow the eyeball protection operation execution notification message to be displayed on a display panel of the image display module.

8. The wearable device of claim 2, wherein the blink control unit is configured to:
receive the temperature sensing signals of the user's left and right eyes in real time through the first and second temperature detection units;
detect temperature values of the user's left and right eyes corresponding to the temperature sensing signals of the user's left and right eyes using a memory or a register;
compare the temperature values of the user's left and right eyes with a threshold temperature value; and
when the temperature values of the user's left and right eyes is greater than the threshold temperature value, transmit spray control signals to the first and second spray driving units.

9. The wearable device of claim 2, wherein the eyeball protection module further comprises a use time detection unit configured to monitor an image display period of a display panel in the image display module and to accumulate and output information on a gaze period during which a user gazes at a displayed image, and
wherein the blink control unit is configured to:
receive the accumulated information on the gaze period during which the user gazes at the displayed image from the use time detection unit and compare the information on the gaze period of the user with use time reference information for each step; and
when the information on the gaze period of the user is the same as the use time reference information for each step, transmit spray control signals to the first and second spray driving unit.

10. The wearable device of claim 2, wherein the lens frame comprises first and second lens frames respectively corresponding to positions of the user's left and right eyes, and
wherein the first and second lens frames are configured to refract the image display light emitted from the image display surface of the image display module in a front direction at an angle in an outer direction or an outer circumferential direction as compared with the front direction and to emit the refracted image display light to rear surfaces of first and second multi-channel lenses, respectively.

11. The wearable device of claim 10, wherein the multi-channel lens comprises first and second multi-channel lenses respectively corresponding to the first and second lens frames, and
wherein the first and second multi-channel lenses are configured to pass the image display light refracted by the first and second multi-channel lenses through different emission paths along the plurality of channels and to transfer the image display light in a user's eyeball disposition direction through a panel of the different emission paths.

12. The wearable device of claim 10, wherein each of the first and second lens frames comprises:
a lens sheet having an area and a shape corresponding to the image display surface of the image display module; and
a plurality of optical lenses on a front surface of the lens sheet and configured to refract the image display light passing through the lens sheet in an outer direction or an outer circumferential direction of the lens sheet and to emit the refracted image display light.

13. A wearable device comprising:
a main frame supporting a transparent lens;
an image display module configured to display an augmented reality content through the transparent lens; and
an eyeball protection module on the main frame and configured to spray air, water, or tear solution to user's left and right eyes.

14. The wearable device of claim 13, wherein the image display module comprises:
an image display device assembled to at least one side of the main frame or formed integrally with the main frame and configured to display an augmented reality content image; and
an image transmission member configured to transmit the augmented reality content image to the transparent lens, and
wherein the augmented reality content image is displayed through reflective members formed in the image transmission member and the transparent lens.

15. The wearable device of claim 14, wherein the image display device comprises:
a partition wall partitioned in a matrix structure on a substrate;
a plurality of light emitting elements, each in a plurality of emission areas arranged in the matrix structure partitioned by the partition wall and extending in a thickness direction of the substrate;
base resins in the plurality of emission areas comprising the plurality of light emitting elements; and
a plurality of optical patterns selectively on at least one of the plurality of emission areas.

16. The wearable device of claim 15, wherein the plurality of emission areas comprises first to third emission areas or first to fourth emission areas in the matrix structure in each pixel area.

17. The wearable device of claim 13, wherein the eyeball protection module comprises:
first and second blink sensing units configured to sense blink motions of the user's left and right eyes in real time and to generate and output blink sensing signals of the user's left and right eyes;
first and second temperature detection units configured to sense temperatures of the user's left and right eyes in real time and to output temperature sensing signals of the user's left and right eyes;
first and second spray driving units configured to spray the air, water, or tear solution to the user's left and right eyes to induce user's blink motions; and
a blink control unit configured to control spray driving of the first and second spray driving units based on at least one of the blink sensing signals of the user's left and right eyes and the temperature sensing signals for the user's left and right eyes.

18. The wearable device of claim 17, wherein the first and second blink sensing unit comprise:
an image sensor configured to capture images of the user's left and right eyes in real time;
an image analyzer configured to compare captured eyeball images with each other in units of at least one frame and to detect eyelid images according to a comparison result; and
a blink sensing signal output unit configured to generate blink sensing signals according to the detection of the eyelid images and to supply the blink sensing signals to the blink control unit.

19. The wearable device of claim 17, wherein the first and second temperature detection units comprise:
a contactless thermometer configured to sense the temperatures of the user's left and right eyes in real time; and
an analog-to-digital converter configured to transmit the temperature sensing signals corresponding to the sensed temperatures of the user's left and right eyes to the blink control unit.

20. The wearable device of claim 17, wherein the first and second spray driving units comprise:
a storage capsule for storing the air, water, or tear solution; and
a spray pump configured to spray the air, water, or tear solution to the user's left and right eyes during a period in which a spray control signal is input from the blink control unit.
